SEQUENCE LISTING (35 sequences)

SEQ ID NO: 1 - DNA, 217 bp, synthetic construct
  source 1..217: mol_type = other DNA
tcgagttccccgcgccagcggggataaaccgaaaagcatataatgcgtaaaagttatgaagttcgagttccccgcgccagcggggataaaccgtattgaccaattcattcgggacagttattagttcgagttccccgcgccagcggggataaaccggtttttgtaattttacaggcaaccttttattcgagttccccgcgccagcggggataaaccg

SEQ ID NO: 2 - DNA, 40 bp, synthetic construct
  source 1..40: mol_type = other DNA
gcgccagcggggataaaccggtttttgtaattttacaggc

SEQ ID NO: 3 - DNA, 28 bp, synthetic construct
  source 1..28: mol_type = other DNA
cttgcccgcctgatgaatgctcatccgg

SEQ ID NO: 4 - DNA, 28 bp, synthetic construct
  source 1..28: mol_type = other DNA
ccggatgagcattcatcaggcgggcaag

SEQ ID NO: 5 - DNA, 44 bp, synthetic construct
  source 1..44: mol_type = other DNA
cggtttatccccgctggcgcggggaactcgaactaataactgtc

SEQ ID NO: 6 - DNA, 76 bp, synthetic construct
  source 1..76: mol_type = other DNA
taacaataaaaatgaaaatgatttccacgatacagaaaaaagagactgtcatcctaatttttgttgacactctatc

SEQ ID NO: 7 - DNA, 75 bp, synthetic construct
  source 1..75: mol_type = other DNA
tgccactcaggtatgatgggcagaatattgcctctgcccgccagaaaaagatcaaagggaaaactgtccatatgc

SEQ ID NO: 8 - DNA, 19 bp, synthetic construct
  source 1..19: mol_type = other DNA
ccgacagggattccatctg

SEQ ID NO: 9 - DNA, 26 bp, synthetic construct
  source 1..26: mol_type = other DNA
tatgacgaccattttgtctacagttc

SEQ ID NO: 10 - DNA, 22 bp, synthetic construct
  source 1..22: mol_type = other DNA
ctggtacacgctgatgaacacc

SEQ ID NO: 11 - DNA, 21 bp, synthetic construct
  source 1..21: mol_type = other DNA
ctggtcattgccatttgtgcc

SEQ ID NO: 12 - DNA, 20 bp, synthetic construct
  source 1..20: mol_type = other DNA
gaatcagagcgttccgaccc

SEQ ID NO: 13 - DNA, 20 bp, synthetic construct
  source 1..20: mol_type = other DNA
gtacgcagtttgccaacgtg

SEQ ID NO: 14 - DNA, 75 bp, synthetic construct
  source 1..75: mol_type = other DNA
aatagcccgctgatatcatcgataatactaaaaaaacagggaggctattatcctaatttttgttgacactctatc

SEQ ID NO: 15 - DNA, 75 bp, synthetic construct
  source 1..75: mol_type = other DNA
tacagggatccagttatcaataagcaaattcatttgttctccttcatatgatcaaagggaaaactgtccatatgc

SEQ ID NO: 16 - DNA, 27 bp, synthetic construct
  source 1..27: mol_type = other DNA
caagacatgtgtatatcactgtaattc

SEQ ID NO: 17 - DNA, 23 bp, synthetic construct
  source 1..23: mol_type = other DNA
gcgattgcagatttatgatttgg

SEQ ID NO: 18 - DNA, 19 bp, synthetic construct
  source 1..19: mol_type = other DNA
gcaaaatgctggctcattg

SEQ ID NO: 19 - DNA, 22 bp, synthetic construct
  source 1..22: mol_type = other DNA
gaactgaatggcaaactgactg

SEQ ID NO: 20 - DNA, 20 bp, synthetic construct
  source 1..20: mol_type = other DNA
tatcatcctgaaagcgatgg

SEQ ID NO: 21 - DNA, 24 bp, synthetic construct
  source 1..24: mol_type = other DNA
caaaagagattctgggtattcact

SEQ ID NO: 22 - DNA, 21 bp, synthetic construct
  source 1..21: mol_type = other DNA
gagcatggtgatcttctcagt

SEQ ID NO: 23 - DNA, 22 bp, synthetic construct
  source 1..22: mol_type = other DNA
gcgatgaatgtcttactacgga

SEQ ID NO: 24 - DNA, 19 bp, synthetic construct
  source 1..19: mol_type = other DNA
gtcgctgggtaatctgcaa

SEQ ID NO: 25 - DNA, 23 bp, synthetic construct
  source 1..23: mol_type = other DNA
atcaacgcatatagcgctagcag

SEQ ID NO: 26 - DNA, 18 bp, synthetic construct
  source 1..18: mol_type = other DNA
actgaagcccagacgatc

SEQ ID NO: 27 - DNA, 707 bp, synthetic construct
  source 1..707: mol_type = other DNA
gcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatattgactaccggaagcagtgtgaccgtgtgcttctcaaatgcctgattcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgat

SEQ ID NO: 28 - DNA, 70 bp, synthetic construct
  source 1..70: mol_type = other DNA
aaatgatttccacgatacagaaaaaagagactgtcatgggcagaatattgcctctgcccgccagaaaaag

SEQ ID NO: 29 - DNA, 70 bp, synthetic construct
  source 1..70: mol_type = other DNA
ctgtttcgatttagttggcaatttaggtagcaaactcggctttaccaccgtcaaaaaaaacggcgctttt

SEQ ID NO: 30 - DNA, 674 bp, synthetic construct
  source 1..674: mol_type = other DNA
taccccgcgagcataaaacgcgtgtgtaggaggataatctatggatttgtcacagctaacaccacgtcgtccctatctgctgcgtgcattctatgagtggttgctggataaccagctcacgccgcacctggtggtggatgtgacgctccctggcgtgcaggttcctatggaatatgcgcgtgacgggcaaatcgtactcaacattgcgccgcgtgctgtcggcaatctggaactggcgaatgatgaggtgcgctttaacgcgcgctttggtggcattccgcgtcaggtttctgtgccgctggctgccgtgctggctatctacgcccgtgaaaatggcgcaggcacgatgtttgagcctgaagctgcctacgatgaagataccagcatcatgaatgatgaagaggcatcggcagacaacgaaaccgttatgtcggttattgatggcgacaagccagatcacgatgatgacactcatcctgacgatgaacctccgcagccaccacgcggtggtcgaccggcattacgcgttgtgaagtaattgacggctagctcagtcctaggtacagtgctagccatatgaaggagaacaaatgaatttgcttattgataactggatccctgtacgcccgcgaaacggggggaaagtccaaatcataaatctgcaatcgctatac

SEQ ID NO: 31 - DNA, 670 bp, synthetic construct
  source 1..670: mol_type = other DNA
cagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaagaagttcctattctctagaaagtataggaacttccgttctgttggtaaagatgggcggcgttctgccgcccgttatctctgttatacctttctgatatttgttatcgccgatccgtctttctccccttcccgccttgcgtcagg

SEQ ID NO: 32 - DNA, 720 bp, synthetic construct
  source 1..720: mol_type = other DNA
gtattccgtcttccatgttcaccgtcattttcgcaatggcacgtaccgttggctggatcgcccactggagcgaaatgcacagtgacggtatgaagattgcccgtccgcgtcagctgtatacaggatatgaaaaacgcgactttaaaagcgatatcaagcgtgcggccaacgatgaaaactattctgaaaactatgcggatgcgtcttaatagttgacaattaatcatcggcatagtatatcggcatagtataatacgactcactataggagggccatcatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcactttgtggcagaggagcaggactgaggataagtaatggttgattgctaagttgtaaatattttaacccgccgttcatatggcgggttgattt

SEQ ID NO: 33 - DNA, 672 bp, synthetic construct
  source 1..672: mol_type = other DNA
ggccaacgatgaaaactattctgaaaactatgcggatgcgtcttaatagttgacaattaatcatcggcatagtatatcggcatagtataatacgactcactataggagggccatcatgaagaccttcaacatctctcagcaggatctggagctggtggaggtcgccactgagaagatcaccatgctctatgaggacaacaagcaccatgtcggggcggccatcaggaccaagactggggagatcatctctgctgtccacattgaggcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgcagagagctcatctctgactatgctcctgactgctttgtgctcattgagatgaatggcaagctggtcaaaaccaccattgaggaactcatccccctcaagtacaccaggaactaaagtaaaactttatcgaaatggccatccattcttgcgcggatggcctctgccagctgctcatagcggctgcgcagcggtgagccaggacgataaaccaggccaatagtgcggcgtggttccggcttaatgcacgg

SEQ ID NO: 34 - DNA, 1651 bp, synthetic construct
  source 1..1651: mol_type = other DNA
cgccgcaaaccccgcccctgacagggcggggtttcgccgcacgtctccatcgcttgcccaagttgtgaagcacagctaacaccacgtcgtccctatctgctgccctaggtctatgagtggttgctggataactttacgggcatgcataaggctcgtatgatatattcagggagaccacaacggtttccctctacaaataattttgtttaactttcgtagaagagcacttccacacttctggaaaaggagatataccatgccagatgccaaaaagcaaggccgttctaacaaggcaatgacattcttcgtgtgcttccttgcggcgcttgccggcctcttgttcggcttggacatcggcgtcattgccggtgctttaccatttatcgctgacgaattccagatcacctcgcacacgcaagaatgggtcgtaagctccatgatgttcggtgcggcagtcggtgcggtgggcagcggctggctctcctttaaactcgggcgcaaaaagagcctgatgatcggcgcaattttgtttgttgccggttcgctgttctctgcggctgcgccaagcgttgaagtactgattctttcccgcgttctactggggctggcggtgggtgtggcctcttataccgcaccgctgtacctctctgaaattgcgccggaaaaaattcgtggcagtatgatctcgatgtatcagttgatgatcactatcgggatcctcggtgcttatctttctgataccgccttcagctacaccggtgcatggcgctggatgctgggtgtgattatcatcccggcaattttgctgctgattggtgtcttcttcctgccagacagcccacgttggtttgccgccaaacgccgttttgttgatgccgaacgcgtgctgctacgcctgcgtgacaccagcgcggaagcgaaacgcgaactggatgaaatccgtgaaagtttgcaggttaaacagagtggctgggcgctgtttaaagagaacagcaacttccgccgcgcggtgttccttggcgtactgttgcaggtaatgcagcaattcaccgggatgaacgtcatcatgtattacgcgccgaaaatcttcgaactggcgggttataccaacactaccgagcaaatgtgggggaccgtgattgtcggcctgaccaacgtacttgccacctttatcgcaatcggccttgttgaccgctggggacgtaaaccaacgctaacgctgggcttcctggtgatggctgctggcatgggcgtactcggtacaatgatgcatatcggtattcactctccgtcggcgcagtatttcgccatcgccatgctgctgatgtttattgtcggttttgccatgagtgccggtccgctgatttgggtactgtgctccgaaattcagccgctgaaaggccgcgattttggcatcacctgctccactgccaccaactggattgccaacatgatcgttggcgcaacgttcctgaccatgctcaacacgctgggtaacgccaacaccttctgggtgtatgcggctctgaacgtactgtttatcctgctgacattgtggctggtaccggaaaccaaacacgtttcgctggaacatattgaacgtaatctgatgaaaggtcgtaaactgcgcgaaataggcgctcacgattaa

SEQ ID NO: 35 - DNA, 1223 bp, synthetic construct
  source 1..1223: mol_type = other DNA
cgccgcaaaccccgcccctgacagggcggggtttcgccgcacgtctccatcgcttgcccaagttgtgaagcacagctaacaccacgtcgtccctatctgctgccctaggtctatgagtggttgctggataactttacgggcatgcataaggctcgtatgatatattcagggagaccacaacggtttccctctacaaataattttgtttaactttcgtagaagagcacttccacacttctggaaaaaggagatataccatgaccaagtatgccctggtcggtgacgtaggtggtaccaatgcacgtctcgctctctgtgatatcgcaagcggggaaatttctcaggccaaaacatattccgggttggattaccccagcttagaagccgtgattcgtgtctatttagaagaacataaagtagaagtcaaagacggttgtattgctattgcgtgccccatcactggggattgggtagcaatgaccaaccatacctgggcgttttctattgccgagatgaaaaaaaatctgggtttctcacacctggagatcatcaacgattttaccgcggtgagcatggcgatcccaatgttaaaaaaggaacacttaattcagttcggcggggcagaacctgtggagggcaagccgatcgcggtttatggtgcaggcacaggcttaggtgtcgcgcacttggtacatgttgacaagcgctgggtgagtttgccgggcgaaggcggccacgtggattttgcccccaattctgaagaggaggcgattattctggaaatcttgcgtgcagaaatcggtcatgtgtctgccgaacgtgtgctgagtggtccaggtctggtgaatctgtaccgcgctattgtcaaagcggataaccgcctgccagaaaaccttaaaccgaaagatatcaccgaacgtgccttggccgactcctgtaccgattgccgccgcgcacttagtctgttttgcgttatcatgggtcgttttggcggcaacctcgcgctgaacctggggacctttggcggtgtttttattgcgggaggtattgttccacgctttttagaatttttcaaagccagtggctttcgcgcggccttcgaagacaagggacgttttaaagaatacgtacatgatatcccagtctatttaattgttcacgataacccaggactgttaggctctggtgcccatctgcgtcagacattgggccatattctgtaa

(19) United States
(12) Patent Application Publication
(10) Pub. No.: US 2025/0270596 A1
(43) Pub. Date: Aug. 28, 2025

(54) MEVALONATE DERIVATIVES AND METHODS FOR MAKING THE SAME

(71) Applicants: Duke University, Durham, NC (US); DMC Biotechnologies, Inc., Boulder, CO (US)

(72) Inventors: Michael David Lynch, Durham, NC (US); Zhixia Ye, Raleigh, NC (US); Moses Onyeabor, Boulder, CO (US); Sal Munoz, Boulder, CO (US)

(21) Appl. No.: 19/086,909

(22) Filed: Mar. 21, 2025

Related U.S. Application Data
(63) Continuation-in-part of application No. 18/317,588, filed on May 15, 2023.
Application No. 18/317,588 is a continuation of application No. 16/487,542, filed on Aug. 21, 2019, now Pat. No. 11,268,111.
(60) Provisional application No. 63/568,103, filed on Mar. 21, 2024.
(60) Provisional application No. 62/461,436, filed on Feb. 21, 2017.

(51) Int. Cl. C12P 7/42; C12N 9/02; C12N 9/04; C12N 9/06; C12N 9/10; C12N 15/74; C12P 13/06

(52) U.S. Cl. CPC C12P 7/42; C12N 9/0006; C12N 9/0008; C12N 9/001; C12N 9/0016; C12N 9/0051; C12N 9/1025; C12N 15/746; C12P 13/06

(57) ABSTRACT

Biofermentation processes giving rise to high-level production of mevalonate are provided, and, in turn, valuable known and novel downstream products are synthesized therefrom.

### CROSS-REFERENCE TO RELATED APPLICATIONS

This application claims the benefit of U.S. Provisional Application No. 63/568,103, filed on Mar. 21, 2024. This application is also a continuation-in-part of U.S. application Ser. No. 18/317,588, filed on May 15, 2023, which is a continuation of U.S. application Ser. No. 16/487,542, filed on Aug. 21, 2019, and now issued as U.S. Pat. No. 11,268,111B2, which is a National Stage Entry of PCT/US2018/019040, filed on Feb. 21, 2018, which claims the benefit of U.S. Provisional Application No. 62/461,436, filed on Feb. 21, 2017. Each of these applications is incorporated herein by reference in its entirety.

### SEQUENCE LISTING

A Sequence Listing has been submitted electronically in XML format and is hereby incorporated by reference in its entirety. The XML copy, created on May 20, 2024, is named 49186-146.xml and is 38,571 bytes in size.

### BACKGROUND

Biotechnology-based fermentation processes have been successfully developed to produce everything from biologics and small molecule therapies to specialty, bulk, and commodity chemicals, and even next generation biofuels. These processes have made rapid advancements in recent years due to technology developments in the fields of fermentation science and synthetic biology, as well as metabolic and enzyme engineering. Despite these substantial advances, most successful examples of rational and directed engineering approaches have also greatly relied on numerous and often lengthy cycles of trial and error.

Mevalonic acid (MVA) (chemical formula C₆H₁₂O₄, IUPAC name (±)-3,5-dihydroxy-3-methylpentanoic acid), characterized by the general structure:

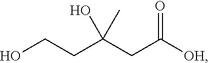

is a precursor substance of the mevalonate pathway, which is essential for cell growth and proliferation. Mevalonate is the carboxylate form of MVA. Metabolic engineering has successfully harnessed the mevalonate pathway to produce a variety of isoprenoids with diverse applications, including as biofuels, fragrances, and pharmaceuticals. MVA is a promising substrate for the sustainable production of various monomers and polymers derived from mevalonolactone (MVL), β-methyl-δ-valerolactone, and anhydromevalonolactone (AMVL). However, mevalonate productivity from microbial fermentations remains unsatisfactory.

What is needed is a biofermentation process giving rise to high-level production of mevalonate, and, in turn, valuable known and novel downstream products derived therefrom.

### SUMMARY

In one such aspect, a method is provided for preparing a mevalonate derivative represented by one of the following formulas:

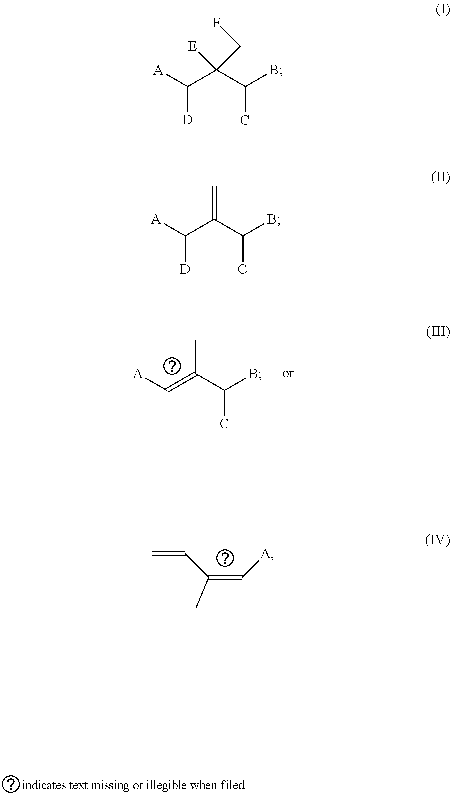

wherein: A and B are combinations of: COOH, COOR, COCl, CONR′R″, COSR, (CO)O(CO)R′, COH, CNR′R″, CN, CX (X=Cl, Br, I), CHO, H, CH3, NCO, CSR; C=Halogen, OH, H, R, NR′R″, O (oxirane), SR, OR; D=Halogen, OH, H, R, NR′R″, O (oxirane), SR, OR; E=Halogen, OH, H, R, NR′R″, O (oxirane), SR, OR; F=Halogen, OH, H, R, NR′R″, O (oxirane), SR, OR; and R, R′, R″ are any combination of =H, alkyl, aryl, carbocycle, the method comprising: providing a genetically modified E. coli for producing mevalonate; growing the genetically modified E. coli in a media in a growth phase, the genetically modified E. coli comprising: a production pathway comprising at least one production enzyme for biosynthesis of mevalonate; and one or more synthetic metabolic valves for reducing or eliminating flux through multiple metabolic pathways within the genetically modified E. coli when the synthetic metabolic valves are induced, the one or more synthetic metabolic valves comprising: at least one silencing synthetic metabolic valve that silences gene expression of two gene encoding two silenceable enzymes that are selected from fabI, gltA, ldp, zwf, or udhA gene, and at least one proteolytic synthetic metabolic valve that controls proteolysis of a fabI, gltA, ldp, zwf, or udhA enzyme; transitioning to a productive stationary phase, the transition comprising: depletion of a limiting nutrient; inducing the one or more synthetic metabolic valves; and activation of the production enzyme for biosynthesis of mevalonate; producing mevalonate; and acidifying mevalonate to provide MVA.

In one such aspect, the method further comprises conversion of MVA to a lactone represented by any one of the following formulas:

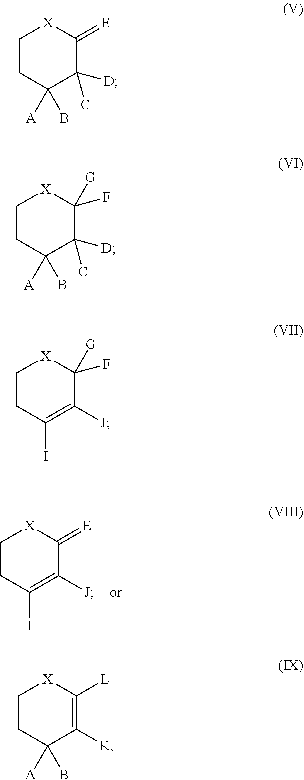

wherein: X=O or N; A, B, C, and D are combinations of H, R, OH, OR, O (oxirane), SR, CRS=O, NR′R″, Halogen; E=O, S, N=R; F and G are combinations of: H, R, CR, OH, OR, O (oxirane), CRS=O, NR′R″, Halogen; I and J are combinations of: H, R, OR, Halogen; K=H, R, OR, Halogen; and L=H, R, CR, OH, OR, CRS=O, NR′R″, Halogen.

### BRIEF DESCRIPTION OF THE FIGURES

FIG. 1 shows metabolic pathways and synthetic metabolic valves (SMVs) for mevalonate production.

FIG. 2 shows mevalonate production using several production pathway plasmid variants with varied promoter combinations in the control strain.

FIG. 3 shows micro-fermentation results for a subset of “Valve” strains producing mevalonate, using the best production pathway from FIG. 2, along with combinations of proteolytic and silencing SMVs.

FIG. 4 shows μL to 1 L scalability for a subset of mevalonate strains evaluated at the 1 L scale. N=3 for μL data and n=1 for 1 L data. The maximal titer within 50 h of production time was used for the correlation.

FIG. 5 shows a Specific Productivity (SP) comparison for a strain with the highest mevalonate titer from literature and mevalonate strain 1 prepared as described herein.

FIG. 6 shows a TUV standard curve for MVA.

FIG. 7 shows an example mevalonate production fermentation plot from FIG. 4 (silencing of fabI-gltA1-gltA2 (FG1G2), proteolysis of fabI and udhA (FU)) in 1 L bioreactors.

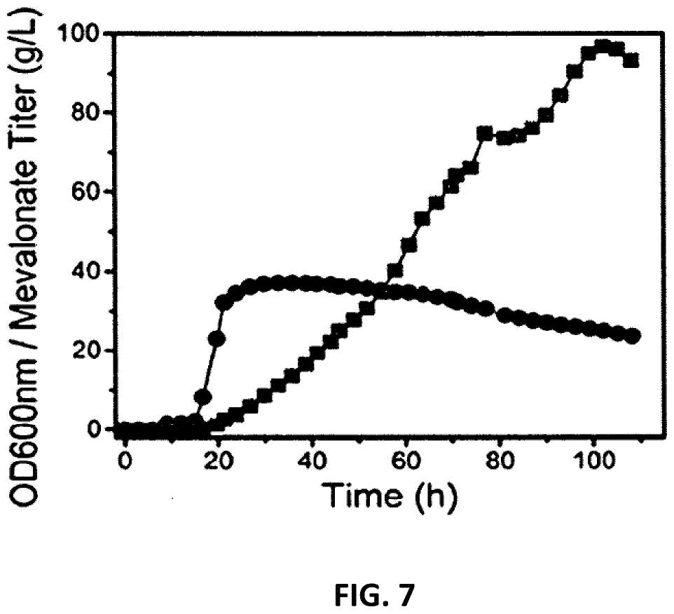

FIG. 8 shows an example mevalonate production feeding/aeration profile.

FIG. 9 shows another example mevalonate production fermentation plot.

FIG. 10 shows an example synthetic route to 3-methyl-1,5-pentanediol (3MPD) from MVA.

FIG. 11 shows an example synthetic route to 3MPD from MVA.

FIG. 12 shows an example synthetic route to 3MPD from MVA.

FIG. 13 shows an example synthetic route to 3MPD from MVA.

FIG. 14 shows an example synthetic route to 3MPD from MVA.

FIG. 15 shows an example synthetic route to 3MPD from MVA.

FIG. 16 shows an example synthetic route to 3-methylpentanedioic acid from MVA.

FIG. 17 shows an example synthetic route to 3-methylpentanedioic acid from MVA.

FIG. 18 shows an example synthetic route to two isomers of 3-methylpentanedioic acid from MVA.

FIG. 19 shows example deoxygenation synthetic schemes from MVA.

FIG. 20 shows example dehydration synthetic schemes from MVA to yield mevalonolactone (MVL) or 5,6-dihydro-4-methyl-2H-pyran-2-one or anhydromevalonolactone (AMVL).

FIG. 21 shows an example substitution reaction from MVL.

FIG. 22 shows an example ring fusion reaction of MVL, followed by lactamization.

FIG. 23 shows an example lactamization reaction of MVL.

FIG. 24 shows an example ring opening reaction of MVL, followed by lactamization.

FIG. 25 shows an example halogenation of AMVL.

FIG. 26 shows example cycloaddition and epoxidation reactions of AMVL.

FIG. 27 shows an example substitution reaction of AMVL, followed by deacetylation.

FIG. 28 shows an example hydrogenation of AMVL to yield β-methyl-δ-valerolactone.

FIG. 29 shows example substitution reactions of β-methyl-δ-valerolactone.

FIG. 30 shows example allylations of β-methyl-δ-valerolactone.

FIG. 31 shows example acetal formation and spiroketal formation reactions of β-methyl-δ-valerolactone.

FIG. 32 shows an example lactamization of β-methyl-δ-valerolactone.

FIG. 33 shows an example thione formation by subjecting β-methyl-δ-valerolactone to Lawesson's Reagent.

### DETAILED DESCRIPTION

### Definitions

The singular forms “a,” “an,” and “the” include plural referents unless the context clearly dictates otherwise. Thus, for example, reference to “an expression vector” includes a single expression vector as well as a plurality of expression vectors, either the same (e.g., the same operon) or different; and reference to “a microorganism” includes a single microorganism as well as a plurality of microorganisms.

The term “about” in conjunction with a number is intended to include ±10% of the number. That is, the number is intended to be read as if it was followed by the phrase, “±10%.” This is true whether “about” is modifying a stand-alone number or modifying a number at either or both ends of a range of numbers. In other words, “about 10” means from 9 to 11. Likewise, “about 10 to about 20” contemplates 9 to 22 and 11 to 18. In the absence of the term “about,” the exact number is intended. In other words, “10” means 10.

The terms “comprising” and “including” are intended to be equivalent and open-ended. The phrase “consisting essentially of” means that the composition or method may include additional ingredients and/or steps, but only if the additional ingredients and/or steps do not materially alter the basic and novel characteristics of the claimed composition or method. The phrase “selected from the group consisting of”' is meant to include mixtures of the listed group.

The terms “controlled” and “regulated” as in “controlled gene silencing” or “controlled proteolysis” means specific activity (e.g., specific removal or modification by proteolytic cleavage of proteins) in response to specific signals (e.g., depending on a high degree of protease substrate specificity).

When reference is made to the term “each,” it is not meant to mean “each and every, without exception.” For example, if reference is made to each of multiple synthetic metabolic valves, and “each synthetic metabolic valve” is to comprise one or more genes for: (i) controlled silencing of gene expression of at least one gene; or (ii) controlled proteolytic inactivation of at least one protein, if there are 10 synthetic metabolic valve, and two or more of the synthetic metabolic valve have such a gene, then that subset of two or more synthetic metabolic valves is intended to meet the limitation.

The term “gene disruption” and prosaic and grammatical equivalents thereof (e.g., “to disrupt enzymatic function,” “disruption of enzymatic function,” and the like), are intended to mean a genetic modification to a microorganism that renders the encoded gene product as having a reduced polypeptide activity compared with polypeptide activity in or from a microorganism cell not so modified. The genetic modification can be, for example, deletion of the entire gene, deletion or other modification of a regulatory sequence required for transcription or translation, deletion of a portion of the gene resulting in a truncated gene product (e.g., a truncated enzyme) or by any of various mutation strategies that reduce activity (including reducing activity to no detectable activity level) of the encoded gene product. A disruption may broadly include a deletion of all or part of the nucleic acid sequence encoding the enzyme, and also includes, but is not limited to, other types of genetic modifications, e.g., introduction of stop codons, frame shift mutations, introduction or removal of portions of the gene, and introduction of a degradation signal, those genetic modifications affecting mRNA transcription levels and/or stability, and altering the promoter or repressor upstream of the gene encoding the enzyme.

The term “gene silencing” means the regulation of gene expression in a cell to prevent the expression of a certain gene. Gene silencing can occur during either transcription or translation. Gene silencing methods may include, for example, RNAi, CRISPR, and siRNA, and generally reduce the expression of a gene by, e.g., 70% or more, but do not eliminate it.

The term “heterologous” includes the term “exogenous” as the latter term is generally used in the art. With reference to the host microorganism's genome prior to the introduction of a heterologous polynucleotide, the nucleic acid sequence that codes for the enzyme is heterologous (whether or not the heterologous polynucleotide is introduced into that genome).

The term “heterologous polynucleotide” or “heterologous gene” as used herein refers to a polynucleotide comprising a nucleic acid sequence wherein at least one of the following is true: (a) the sequence of nucleic acids is foreign to (i.e., not naturally found in) a given host microorganism; (b) the sequence may be naturally found in a given host microorganism, but in an unnatural (e.g., greater than expected) amount; or (c) the sequence of nucleic acids comprises two or more subsequences that are not found in the same relationship to each other in nature. For example, regarding instance (c), a heterologous polynucleotide that is recombinantly produced will have two or more nucleic acid sequences from unrelated genes arranged to make a new functional nucleic acid, such as a nonnative promoter driving gene expression.

The term “metabolic flux” refers to changes in metabolism that lead to changes in product and/or byproduct formation, including production rates, production titers, and production yields from a given substrate.

The term “mutant” can refer to an “amino acid modification,” including an amino acid substitution, insertion, or deletion in a polypeptide sequence, or a “mutant gene,” including a substitution, insertion, or deletion in a nucleic acid sequence. By “amino acid substitution” or “substitution” is meant the replacement of an amino acid at a particular position in a parent polypeptide sequence with another amino acid. For example, the substitution R94K refers to a modified polypeptide in which the arginine at position 94 is replaced with a lysine. Multiple substitutions may be separated by a slash or a comma. For example, R94K/L78V and [R94K, L78V] refer to a double variant comprising the substitutions R94K and L78V. By “amino acid insertion” or “insertion” is meant the addition of an amino acid at a particular position in a parent polypeptide sequence, including an addition to a C- or N-terminus. For example, insert −94 designates an insertion at position 94. By “amino acid deletion” or “deletion” is meant the removal of an amino acid at a particular position in a parent polypeptide sequence. For example, R94−designates the deletion of arginine at position 94. Similarly, in the case of a gene mutant, A110G refers to a substitution of an adenine at position 110 with a guanine.

The term “proteolysis” means the breakdown of proteins into smaller polypeptides or amino acids. Proteolysis may be catalyzed by cellular enzymes known as proteases or by intra-molecular digestion.

The phrases “reduced enzymatic activity,” “reducing enzymatic activity,” and the like are meant to indicate that a microorganism cell or an isolated enzyme exhibits a lower level of activity than that measured in a comparable non-modified cell of the same species or its native (non-modified) enzyme. That is, enzymatic conversion of the indicated substrate(s) to indicated product(s) under known standard conditions for that enzyme is at least 10, at least 20, at least 30, at least 40, at least 50, at least 60, at least 70, at least 80, or at least 90 percent less than the enzymatic activity for the same biochemical conversion by a native enzyme under a standard specified condition. This term also can include elimination of that enzymatic activity. A cell having reduced enzymatic activity of an enzyme can be identified using any method known in the art. For example, enzyme activity assays can be used to identify cells having reduced enzyme activity. See, for example, Enzyme Nomenclature, Academic Press, Inc., New York 2007.

The term “synthetic metabolic valve” or “SMV” refers either to the use of controlled proteolysis, gene silencing, or the combination of both proteolysis and gene silencing to alter metabolic fluxes.

The meaning of abbreviations is as follows: “C” means Celsius or degrees Celsius, as is clear from its usage; DCW means dry cell weight; “s” means second(s); “min” means minute(s); “h,” “hr,” or “hrs” means hour(s); “psi” means pounds per square inch; “nm” means nanometers; “d” means day(s); “μL” or “uL” or “ul” means microliter(s); “mL” means milliliter(s); “L” means liter(s); “mm” means millimeter(s); “nm” means nanometers; “mM” means millimolar; “μM” or “uM” means micromolar; “M” means molar; “mmol” means millimole(s); “μmol” or “umol” means micromole(s); “g” means gram(s); “μg” or “ug” means microgram(s); “ng” means nanogram(s); “PCR” means polymerase chain reaction; “OD” means optical density; “OD₆₀₀” means the optical density measured at a photon wavelength of 600 nm; “kDa” means kilodaltons; “g” means the gravitation constant; “bp” means base pair(s); “kbp” means kilobase pair(s); “% w/v” means weight/volume percent; “% v/v” means volume/volume percent; “IPTG” means isopropyl-μ-D-thiogalactopyranoiside; “aTc” means anhydrotetracycline; “RBS” means ribosome binding site; “rpm” means revolutions per minute; “HPLC” means high performance liquid chromatography; and “GC” means gas chromatography.

The recitations of numerical ranges by endpoints include all numbers subsumed within that range (e.g., 1 to 5 includes 1, 1.5, 2, 2.75, 3, 3.80, 4, 5, etc.).

When the genetic modification of a gene product, e.g., an enzyme, is referred to herein, including the claims, it is understood that the genetic modification is of a nucleic acid sequence, such as or including the gene, that normally encodes the stated gene product.

Species and other phylogenic identifications are according to the classification known to a person skilled in the art of microbiology.

Enzymes are listed with reference to a Universal Protein Resource (Uniprot) identification number, which would be well known to one skilled in the art (Uniprot is maintained by and available through the UniProt Consortium).

Where methods and steps described herein indicate certain events occurring in certain order, those of ordinary skill in the art will recognize that the ordering of certain steps may be modified, and that such modifications are in accordance with the variations of the invention. Additionally, certain steps may be performed concurrently in a parallel process when possible, as well as performed sequentially.

Bio-production or fermentation, as used herein, may be aerobic, microaerobic, or anaerobic.

The disclosure may refer to genes and proteins for which it provides an example “SEQ ID NO:.” Unless otherwise apparent from the context, reference to a gene or protein should be understood as including the specific SEQ ID NO, as well as allelic, species, and induced variants thereof having at least 90, 95, or 99% identity thereto. Examples of allelic and species variants can be found in the SwissProt and other databases.

### Overview

Provided herein is a high-throughput metabolic engineering platform enabling the rapid optimization of microbial production strains. The platform, which bridges a gap between current in vivo and in vitro bio-production approaches, relies on dynamic minimization of the active metabolic network. Dynamic metabolic network minimization can be accomplished using combinations of CRISPR interference and controlled proteolysis to reduce the activity of multiple enzymes in essential central metabolism. Minimization can be implemented in the context of standardized 2-stage bio-processes. This approach not only can result in a design space with greatly reduced complexity, but also in increased metabolic fluxes and production rates as well as in strains which are robust to environmental conditions. Robustness can lead to predictable scalability from high-throughput small-scale screens, or “micro-fermentations,” to fully instrumented bioreactors. Predictive high-throughput approaches may be critical for metabolic engineering programs to truly take advantage of the rapidly increasing throughput and decreasing costs of synthetic biology. The examples demonstrate rapid optimization of E. coli strains for producing mevalonate at commercially meaningful rates, titers (e.g., 97 g/L), and yields.

### Synthetic Metabolic Valves (SMVs)

In one aspect, SMVs are provided, the SMVs comprising either or both of controlled gene silencing and controlled proteolysis. The development of platform microbial strains that use SMVs can decouple growth from product formation. These strains enable the dynamic control of metabolic pathways, including those that when altered have negative effects on microorganism growth. Dynamic control over metabolism is accomplished via a combination of methodologies including but not limited to transcriptional silencing and controlled enzyme proteolysis. These microbial strains are used in a multi-stage bioprocess encompassing at least two stages: (i) in the first stage, microorganisms are grown, and metabolism can be optimized for microbial growth; and (ii) in at least one other stage, microorganism growth can be slowed or stopped, and dynamic changes can be made to metabolism to improve production of mevalonate or a derivative thereof. The transition of growing cultures between stages and the manipulation of metabolic fluxes can be controlled by artificial chemical inducers or tuning the level of key limiting nutrients. In addition, genetic modifications may be made to provide metabolic pathways for the biosynthesis of mevalonate or a derivative thereof. Also, genetic modifications may be made to enable the use of a variety of carbon feedstocks, including but not limited to sugars, such as glucose, sucrose, xylose, arabinose, mannose, and lactose, as well as oils, carbon dioxide, carbon monoxide, methane, methanol, ethanol, and formaldehyde.

This approach allows for simpler models of metabolic fluxes and physiological demands during a production phase, turning a growing cell into a stationary phase biocatalyst. The SMVs can be used to turn off essential genes and redirect carbon, electrons, and energy flux to mevalonate or a derivative thereof in a multi-stage fermentation process. One or more of the following enables these SMVs: (1) transcriptional gene silencing or repression technologies; (2) inducible enzyme degradation; and (3) nutrient limitation to induce a stationary or non-dividing cellular state. SMVs are generalizable to any pathway and microbial host.

In various cases, one SMV can refer to the manipulation of one gene (or its protein product). The manipulation can be controlled silencing of the gene and/or controlled degradation of its protein product. In certain cases, combination of SMVs can lead to improved production in yields, rate, and/or robustness, which includes manipulation of two or more genes (or their protein products).

### Methods and Systems for Bio-Production

Provided herein are methods and systems for robust large-scale production of mevalonate and derivatives thereof. The methods and systems comprise using engineered microorganisms that comprise metabolic enzymes. In some aspects, the engineered microorganisms comprise at least one metabolic enzyme that has a reduced level or activity. The methods and systems include reduced metabolic responses to environmental conditions and can be easily transferred from small scale (e.g., mg) production to large scale (e.g., kg) production. The methods and systems reduce the time and costs associated with transitioning from small scale to large scale production.

The engineered microorganisms may include genetically modified microorganisms, wherein the microorganisms are capable of producing mevalonate or a derivative thereof derived from any key metabolic intermediate, including but not limited to, malonyl-CoA, pyruvate, oxaloacetate, erthyrose-4-phosphate, xylulose-5-phosphate, alpha-ketoglutarate, and citrate at a specific rate selected from the rates of greater than 0.05 g/gDCW-hr, 0.08 g/gDCW-hr, greater than 0.1 g/gDCW-hr, greater than 0.13 g/gDCW-hr, greater than 0.15 g/gDCW-hr, greater than 0.175 g/gDCW-hr, greater than 0.2 g/gDCW-hr, greater than 0.25 g/gDCW-hr, greater than 0.3 g/gDCW-hr, greater than 0.35 g/gDCW-hr, greater than 0.4 g/gDCW-hr, greater than 0.45 g/gDCW-hr, or greater than 0.5 g/gDCW-hr.

In various aspects, a culture system is provided comprising a carbon source in an aqueous medium and a genetically modified microorganism, wherein the genetically modified organism is present in an amount selected from greater than 0.05 gDCW/L, 0.1 gDCW/L, greater than 1 gDCW/L, greater than 5 gDCW/L, greater than 10 gDCW/L, greater than 15 gDCW/L, or greater than 20 gDCW/L, such as when the volume of the aqueous medium is selected from greater than 5 mL, greater than 100 mL, greater than 0.5 L, greater than 1 L, greater than 2 L, greater than 10 L, greater than 250 L, greater than 1000 L, greater than 10,000 L, greater than 50,000 L, greater than 100,000 L, or greater than 200,000 L, and such as when the volume of the aqueous medium is greater than 250 L and contained within a steel vessel.

### Carbon Sources

Bio-production media, which is used herein with recombinant microorganisms, must contain suitable carbon sources or substrates for both growth and production stages. Suitable substrates may include, but are not limited to, sugars, such as glucose, sucrose, xylose, mannose, and arabinose, as well as oils, carbon dioxide, carbon monoxide, methane, methanol, ethanol, formaldehyde, and glycerol.

### Microorganisms

Suitable host cells or host microorganisms for bio-production can be either prokaryotic or eukaryotic. Suitable host cells or host microorganisms can be bacteria, such as Citrobacter, Enterobacter, Clostridium, Klebsiella, Aerobacter, Lactobacillus, Aspergillus, Saccharomyces, Schizosaccharomyces, Zygosaccharomyces, Pichia, Kluyveromyces, Candida, Hansenula, Debaryomyces, Mucor, Torulopsis, Methylobacter, Escherichia, Salmonella, Bacillus, Streptomyces, and Pseudomonas. In some aspects, a host cell or an engineered cell is E. coli. In some aspects, a host cell or an engineered cell is S. cerevisiae.

In certain aspects, provided herein is a microorganism genetically modified to comprise: a production pathway comprising at least one enzyme for the biosynthesis of mevalonate or a derivative thereof, and a combination of multiple SMVs to controllably reduce or eliminate flux through multiple metabolic pathways. In some aspects, each of the multiple SMVs comprises one or more genes for: (i) controlled silencing of gene expression of at least one gene; or (ii) controlled proteolytic inactivation of at least one protein. In some aspects, a rate of the biosynthesis of mevalonate or a derivative thereof is increased in a productive stationary phase upon a depletion of a nutrient, wherein the depletion of the nutrient induces the multiple SMVs. In some cases, the controlled silencing of gene expression is accomplished by RNA interference, CRISPR interference, or transcriptional repression. In some cases, the controlled proteolytic inactivation is accomplished by protein cleavage by a specific protease or targeted degradation by specific peptide tags. In some cases, the nutrient is phosphate, nitrogen, sulfur, magnesium, or a combination thereof.

In certain aspects, provided herein is a genetically modified microorganism comprising: a production pathway comprising at least one enzyme for the biosynthesis of mevalonate or a derivative thereof from one of the following metabolites: pyruvate, acetolactate, acetyl-CoA, acetoacetyl-CoA, or malonyl-CoA; and a combination of multiple SMVs, wherein each of the multiple SMVs comprises one of a fabI, zwf, lpd, gltA, udhA, or gapA gene for: (i) controlled silencing of gene expression of a corresponding one of such fabI, gltA, lpd, zwf, udhA, or gapA genes; or (ii) controlled proteolytic inactivation of a protein encoded by a corresponding one of such fabI, gltA, lpd, zwf, udhA, or gapA genes. In some aspects, a rate of the biosynthesis of mevalonate or a derivative thereof is increased in a productive stationary phase upon a depletion of a nutrient, wherein the depletion of the nutrient induces the multiple SMVs. In some aspects, the nutrient is phosphate, nitrogen, sulfur, magnesium, or a combination thereof.

In certain aspects, provided herein is a genetically modified microorganism comprising: a production pathway to produce mevalonate or a derivative thereof; and a combination of multiple SMVs, wherein each of the multiple SMVs comprises one of a fabI, gltA, lpd, zwf, udhA, or gapA gene for: (i) controlled silencing of gene expression of a corresponding one of such fabI, gltA, lpd, zwf, udhA, or gapA genes; or (ii) controlled proteolytic inactivation of a protein encoded by one of such fabI, gltA, lpd, zwf, udhA, or gapA genes. In some aspects, a rate of the biosynthesis of mevalonate or a derivative thereof is increased in a productive stationary phase upon a depletion of a nutrient, wherein the depletion of the nutrient induces the multiple SMVs. In some aspects, the nutrient is phosphate, nitrogen, sulfur, magnesium, or a combination thereof.

In some cases, a genetically modified microorganism is a heterologous cell. In some cases, provided herein is a heterologous cell for producing mevalonate or a derivative thereof. In some cases, a heterologous cell comprises an engineered valve polynucleotide for mediating controlled reduction of expression of a valve enzyme acting in a metabolic pathway. In certain cases, a controlled reduction of expression of a valve enzyme reduces flux through a metabolic pathway, wherein the controlled reduction of expression of the valve enzyme induces a stationary phase of the heterologous cell. In some cases, a heterologous cell further comprises an engineered production polynucleotide for mediating controlled increase in expression of a production enzyme for producing mevalonate or a derivative thereof. In some situations, a heterologous cell comprises an engineered valve polynucleotide for mediating controlled reduction of expression of a valve enzyme acting in a metabolic pathway, wherein a rate of production of mevalonate or a derivative thereof during a stationary phase is reduced less in response to a change of an environmental condition as compared to a cell lacking the controlled reduction of expression of the valve enzyme.

In some cases, provided herein is a heterologous cell for producing mevalonate or a derivative thereof, wherein the cell comprises: an engineered valve polynucleotide for mediating controlled reduction of expression of a valve enzyme acting in a metabolic pathway, wherein the controlled reduction of expression of the valve enzyme reduces flux through the metabolic pathway, wherein the controlled reduction of expression of the valve enzyme induces a stationary phase of the cell; and an engineered production polynucleotide for mediating controlled increase in expression of a production enzyme for production of mevalonate or a derivative thereof.

In some cases, provided herein is a cell comprising a reduced expression or activity of a valve enzyme, wherein the valve enzyme comprises an enzyme selected from the group consisting of fabI, zwf, lpd, gltA, udhA, or gapA, and a combination thereof.

In some cases, provided herein is a cell comprising a production enzyme, wherein the production enzyme comprises an enzyme selected from the group consisting of NADPH-dependent alanine dehydrogenase (ald), alanine exporter (alaE), or NADPH-dependent glyceraldehyde-3-phosphate dehydrogenase (gapN), and a combination thereof.

### Environmental Conditions

Environmental conditions can comprise medium and culture conditions. Environmental factors that may influence production can include temperature, pH, acidity, presence of ethanol, presence of sulfite, and availability of nutrients.

In addition to an appropriate carbon source, bio-production media may contain suitable minerals, salts, cofactors, buffers, and other components, known to those skilled in the art, suitable for the growth of the cultures and promotion of the enzymatic pathway necessary for bio- production of mevalonate or a derivative thereof.

Typically, cells are grown at a temperature in the range of about 25° C. to about 40° C. in an appropriate medium, as well as up to 70° C. for thermophilic microorganisms.

Suitable pH ranges for the bio-production are between pH 2.0 to pH 10.0, where pH 6.0 to pH 8.0 is a typical pH range for the initial condition.

Bio-productions may be performed under aerobic, microaerobic, or anaerobic conditions, with or without agitation.

In some cases, a change of an environmental condition comprises a change, e.g., an increase or a decrease, in sugar concentration of a culture medium contacting a cell. In some situations, an increase of sugar concentration is from 1% to 2%, from 2% to 3%, from 3% to 4%, from 4% to 5%, from 5% to 10%, from 10% to 15%, from 15% to 20%, from 20% to 30%, from 30% to 40%, from 40% to 50%, from 50% to 60%, from 60% to 70%, from 70% to 80%, from 80% to 90%, or from 90% to 100% more sugar compared with the original sugar concentration in the culture medium. In some situations, a decrease of sugar concentration is from 1% to 2%, from 2% to 3%, from 3% to 4%, from 4% to 5%, from 5% to 10%, from 10% to 15%, from 15% to 20%, from 20% to 30%, from 30% to 40%, from 40% to 50%, from 50% to 60%, from 60% to 70%, from 70% to 80%, from 80% to 90%, or from 90% to 100% less sugar compared with the original sugar concentration in the culture medium.

In some cases, a change of an environmental condition comprises a change (e.g., an increase or a decrease) in oxygenation of a culture medium contacting a cell. In some situations, an increase of oxygenation is the addition of oxygen from 1% to 2%, from 2% to 3%, from 3% to 4%, from 4% to 5%, from 5% to 10%, from 10% to 15%, from 15% to 20%, from 20% to 30%, from 30% to 40%, from 40% to 50%, from 50% to 60%, from 60% to 70%, from 70% to 80%, from 80% to 90%, or from 90% to 100% more than the original amount of oxygen added in a culture medium. In some situations, a decrease of oxygenation is the addition of oxygen from 1% to 2%, from 2% to 3%, from 3% to 4%, from 4% to 5%, from 5% to 10%, from 10% to 15%, from 15% to 20%, from 20% to 30%, from 30% to 40%, from 40% to 50%, from 50% to 60%, from 60% to 70%, from 70% to 80%, from 80% to 90%, or from 90% to 100% less than the original amount of oxygen added in a culture medium.

### Bio-Production Reactors and Systems

In one aspect, fermentation systems are provided. Any of the recombinant microorganisms as described and/or referred to herein may be introduced into an industrial bio-production system where the microorganisms convert a carbon source into a product in a commercially viable operation. The bio-production system includes the introduction of such a recombinant microorganism into a bioreactor vessel, with a carbon source substrate and bio-production media suitable for growing the recombinant microorganism and maintaining the bio-production system within a suitable temperature range (and dissolved oxygen concentration range if the reaction is aerobic or microaerobic) for a suitable time to obtain a desired conversion of a portion of the substrate molecules to mevalonate or a derivative thereof. Bio-productions may be performed under aerobic, microaerobic, or anaerobic conditions, with or without agitation. The amount of a product produced in a bio-production media generally can be determined using, for example, high performance liquid chromatography (HPLC), gas chromatography (GC), or GC/Mass Spectroscopy (MS).

### Genetic Modifications, Nucleotide Sequences, and Amino Acid Sequences

Aspects of the present disclosure may result from introduction of a heterologous expression vector into a host microorganism, wherein the expression vector comprises a nucleic acid sequence coding for an enzyme that is, or is not, normally found in the host microorganism.

The ability to genetically modify a host cell is essential for the production of any genetically modified (recombinant) microorganism. The mode of gene transfer technology may be by electroporation, conjugation, transduction, or natural transformation. A broad range of host conjugative plasmids and drug resistance markers are available. The cloning vectors are tailored to the host organisms based on the nature of antibiotic resistance markers that can function in that host. Also, as disclosed herein, a genetically modified (recombinant) microorganism may comprise modifications other than via plasmid introduction, including modifications to its genomic DNA.

More generally, nucleic acid constructs can be prepared comprising an isolated polynucleotide encoding a polypeptide having enzyme activity, the isolated polynucleotide operably linked to one or more control sequences that direct the expression of the coding sequence in a microorganism, such as E. coli, under conditions compatible with the control sequences. The isolated polynucleotide may be manipulated to provide for expression of the polypeptide. Manipulation of the polynucleotide's sequence prior to its insertion into a vector may be desirable or necessary depending on the expression vector.

The control sequence may be an appropriate promoter sequence, a nucleotide sequence that is recognized by a host cell for expression of a polynucleotide encoding a polypeptide. The promoter sequence may contain transcriptional control sequences that mediate the expression of the polypeptide. The promoter may be any nucleotide sequence that shows transcriptional activity in the host cell of choice including mutant, truncated, and hybrid promoters, and may be obtained from genes encoding extracellular or intracellular polypeptides either homologous or heterologous to the host cell.

For various aspects, the genetic manipulations may include those directed to change regulation of, and therefore ultimate activity of, an enzyme or enzymatic activity of an enzyme identified in any of the respective pathways. Such genetic modifications may be directed to transcriptional, translational, and post-translational modifications that result in a change of, e.g., enzyme activity and selectivity under selected culture conditions. Such genetic modifications may be directed to the provision of additional nucleic acid sequences such as to increase copy number and the provision of mutants of an enzyme related to product production.

In various aspects, to function more efficiently, a microorganism may comprise one or more gene deletions. For example, in E. coli, the genes encoding the lactate dehydrogenase (IdhA), phosphate acetyltransferase (pta), pyruvate oxidase (poxB), pyruvateformate lyase (pflB), methylglyoxal synthase (mgsA), acetate kinase (ackA), alcohol dehydrogenase (adhE), the clpXP protease specificity enhancing factor (sspB), the ATPdependent Lon protease (Ion), the outer membrane protease (ompT), the arcA transcriptional dual regulator (arcA), and the iclR transcriptional regulator (iclR) may be disrupted, including deleted. Such gene disruptions, including deletions, are not meant to be limiting, and may be implemented in various combinations in various aspects.

In various aspects, to function more efficiently, a microorganism may comprise one or more SMVs, comprised of enzymes targeted for controlled proteolysis, expression silencing, or a combination of both. In some aspects, a microorganism may comprise two or more SMVs. For example, one enzyme encoded by one gene or a combination of numerous enzymes encoded by numerous genes in E. coli may be designed as SMVs to alter metabolism and improve product formation. Representative genes in E. coli may include, but are not limited to, fabl, zwf gltA, ppc, udhA, Ipd, sucD, aceA, pfkA, Ion, rpoS, tktA, or tktB.

For all nucleic acid and amino acid sequences provided herein, conservatively modified variants of these sequences are included. Functionally equivalent nucleic acid and amino acid sequences (functional variants), which may include conservatively modified variants as well as more extensively varied sequences, which are well within the skill of the person of ordinary skill in the art, and microorganisms comprising these, also are included, as are methods and systems comprising such sequences and/or microorganisms.

Accordingly, some compositions, methods, and systems comprise providing a genetically modified microorganism that comprises both a production pathway to make mevalonate or a derivative thereof from a central intermediate in combination with SMVs to redistribute flux.

Some aspects regard provision of multiple genetic modifications to improve microorganism overall effectiveness in converting a selected carbon source into mevalonate or a derivative thereof. Particular combinations are shown, such as in the Examples, to increase specific productivity, volumetric productivity, titer, and yield. In addition, in various aspects, genetic modifications, including SMVs, also are provided to increase the pool and availability of the cofactor NADPH and/or NADH, which may be consumed in the production of mevalonate or a derivative thereof.

More generally, and depending on the particular metabolic pathways of a microorganism selected for genetic modification, any subgroup of genetic modifications may be made to decrease cellular production of fermentation product(s) other than the desired fermentation product, such decreased fermentation product(s) selected from the group consisting of acetate, acetoin, acetone, acrylic, malate, fatty acid ethyl esters, isoprenoids, glycerol, ethylene glycol, ethylene, propylene, butylene, isobutylene, ethyl acetate, vinyl acetate, other acetates, 1,4-butanediol, 2,3-butanediol, butanol, isobutanol, sec-butanol, butyrate, isobutyrate, 2-OH-isobutryate, 3-OH-butyrate, ethanol, isopropanol, D-lactate, L-lactate, pyruvate, itaconate, levulinate, glucarate, glutarate, caprolactam, adipic acid, propanol, isopropanol, fusel alcohols, 1,2-propanediol, 1,3-propanediol, formate, fumaric acid, propionic acid, succinic acid, valeric acid, maleic acid, and poly-hydroxybutyrate. Gene deletions may be made as disclosed generally herein, and other approaches may also be used to achieve a desired decreased cellular production of selected fermentation products other than the desired products.

### Gene Silencing

In one aspect, the use of controlled gene silencing is demonstrated to help enable the control over metabolic fluxes in controlled multi-stage fermentation processes. There are several methodologies known in the art for controlled gene silencing, including but not limited to mRNA silencing or RNA interference, silencing via transcriptional repressors, and CRISPR interference.

In some cases, a valve polynucleotide comprises a polynucleotide selected from the group consisting of: a silencing polynucleotide for repressing transcription of a gene encoding the valve enzyme; a degradation polynucleotide for mediating cellular degradation of the valve enzyme; and a combination thereof.

In some cases, a valve polynucleotide comprises a silencing polynucleotide, and the silencing polynucleotide comprises a guide RNA (gRNA) comprising a gRNA sequence that recognizes a promoter of a gene encoding the valve enzyme.

In some cases, a valve polynucleotide further encodes a CRISPR enzyme, wherein the CRISPR enzyme specifically binds to the promoter sequence when bound to the gRNA. In some cases, a CRISPR enzyme is catalytically inactive.

In some cases, a valve polynucleotide comprises a degradation polynucleotide, wherein the degradation polynucleotide comprises a sequence encoding a degradation tag, wherein the degradation tag mediates degradation of the valve enzyme. In some cases, the expression of a valve polynucleotide is regulated by phosphate availability in a cell. In some cases, the expression of a production polynucleotide is regulated by phosphate availability in a cell. In certain cases, the cell is an E. coli cell.

### Controlled Proteolysis

In one aspect, the use of controlled protein degradation or proteolysis is demonstrated to help enable the control over metabolic fluxes in controlled multi-stage fermentation processes. There are several methodologies known in the art for controlled protein degradation, including but not limited to targeted protein cleavage by a specific protease and controlled targeting of proteins for degradation by specific peptide tags. For example, the E. coli clpXP protease for controlled protein degradation may be suitable. This methodology relies upon adding a specific C-terminal peptide tag such as a DAS4 (or DAS+4) tag. Proteins with this tag are not degraded by the clpXP protease until the specificity enhancing chaperone sspB is expressed. sspB induces degradation of DAS4 tagged proteins by the clpXP protease. Additional site-specific protease systems are well known in the art. Proteins can be engineered to contain a specific target site of a given protease, and the protein cleaved is cleaved at that site upon the controlled expression of the protease. In some aspects, the cleavage leads to protein inactivation or degradation. For example, an N-terminal sequence can be added to a protein of interest to enable clpS dependent clpAP degradation. In addition, this sequence can further be masked by an additional N-terminal sequence, which can be controllably cleaved such as by a ULP hydrolase. This allows for controlled N-end rule degradation dependent on hydrolase expression. It is therefore possible to tag proteins for controlled proteolysis either at the N-terminus or C-terminus.

The preference of using an N-terminal vs. C-terminal tag will largely depend on whether either tag affects protein function prior to the controlled onset of degradation. In one aspect, the use of controlled protein degradation or proteolysis is demonstrated to help enable the control over metabolic fluxes in controlled multi-stage fermentation processes, in E. coli. There are several methodologies known in the art for controlled protein degradation in other microbial hosts, including a wide range of gram-negative as well as gram-positive bacteria, yeast, and even archaea. In one aspect, systems for controlled proteolysis can be transferred from a native microbial host and used in a non-native host.

### SMV Control

In one aspect, the use of SMVs is demonstrated to control metabolic fluxes in multi-stage fermentation processes. There are numerous methodologies known in the art to induce expression that can be used at the transition between stages in multistage fermentations. These include, but are not limited to, artificial chemical inducers, including, for example, tetracycline, anhydrotetracycline, lactose, IPTG (isopropyl-beta-D-1-thiogalactopyranoside), arabinose, raffinose, and tryptophan. Systems linking the use of these inducers to the control of gene expression silencing and/or controlled proteolysis can be integrated into genetically modified microbial systems to control the transition between growth and production phases in multi-stage fermentation processes.

In addition, it may be desirable to control the transition between growth and production in multi-stage fermentations by the depletion of one or more limiting nutrients that are consumed during growth. Limiting nutrients may include, e.g., phosphate, nitrogen, sulfur, and magnesium. Natural gene expression systems that respond to these nutrient limitations can be used to operably link the control of gene expression silencing and/or controlled proteolysis to the transition between growth and production phases in multi-stage fermentation processes.

### Robustness

Robustness can lead to predictable scalability from high-throughput small-scale screens, or “micro-fermentations”, to fully instrumented bioreactors. Predictive high-throughput approaches may be critical for metabolic engineering programs to truly take advantage of the rapidly increasing throughput and decreasing costs of synthetic biology. The examples provided herein have not only demonstrated proof of principle for this approach in the common industrial microbe: E. coli, and has validated this approach with the rapid optimization of E. coli strains producing two important industrial chemicals: mevalonic acid, at commercially meaningful rates, titers (97 g/L).

A lack of environmental robustness is traditionally one factor making the scale up of fermentation based processes difficult. This issue has led to the development of specialized complex micro-reactor systems for scale down offering only modest improvements in throughput.

A central hypothesis was that by restricting metabolism in the production stage, strain performance could not only be improved, but would be more robust to environmental (process) conditions. Robustness scores can be calculated for any subset of product producing “Valve” strains and compared. Simply put, carbon flow is restricted through a minimized metabolic network, which can no longer adapt via cellular responses to the environment. To test this hypothesis, strains were evaluated under different “micro-fermentation” process conditions. Whereas relative standard deviation (RSD) is one metric for robustness, we wanted to incorporate a stricter measure of robustness which also incorporates the maximal deviation (Max Dev) a strain has under all process conditions (RS, Equation (1)).

$$R S = 100 - \frac{average ( R S D ) + max ( Dev )}{2} * 100 \qquad Equation ( 1 )$$

### Fermentation Products

In some aspects, provided herein is a microorganism or a cell for producing mevalonate or a derivative thereof. In some cases, the product comprises mevalonic acid. In some cases, a rate of production of mevalonic acid is at least 0.1 g/L/hr, 0.2 g/L/hr, 0.3 g/L/hr, 0.4 g/L/hr, 0.5 g/L/hr, 0.6 g/L/hr, 0.7 g/L/hr, 0.8 g/L/hr, 0.9 g/L/hr, 1.0 g/L/hr, 1.1 g/L/hr, 1.2 g/L/hr, 1.3 g/L/hr, 1.4 g/L/hr, 1.5 g/L/hr, 1.6 g/L/hr, 1.7 g/L/hr, 1.8 g/L/hr, 1.9 g/L/hr, 2.0 g/L/hr, 2.5 g/L/hr, 3.0 g/L/hr, 3.5 g/L/hr, 4.0 g/L/hr, 4.5 g/L/hr, 5.0 g/L/hr, 5.5 g/L/hr, 6.0 g/L/hr, 7.0 g/L/hr, 8.0 g/L/hr, 9.0 g/L/hr, or at least 10 g/L/hr.

### Synthesis of Mevalonate Derivatives

The mevalonate produced by the biosynthesis process described herein may be acidified to form MVA and transformed into a host of novel and known intermediates and products. In one such aspect, the product may be represented by Formula I:

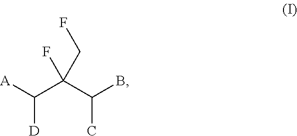

wherein:
A and B are combinations of: COOH, COOR, COCl, CONR′R″, COSR, (CO)O(CO)R′, COH, CNR′R″, CN, CX (X=Cl, Br, I), CHO, H, CH₃, NCO, CSR;
C=Halogen, OH, H, R, NR′R″, O (oxirane), SR, OR;
D=Halogen, OH, H, R, NR′R″, O (oxirane), SR, OR;
E=Halogen, OH, H, R, NR′R″, O (oxirane), SR, OR;
F=Halogen, OH, H, R, NR′R″, O (oxirane), SR, OR; and
R, R′, R″ are any combination of =H, alkyl, aryl, carbocycle.

In another such aspect, the product may be represented by Formula II:

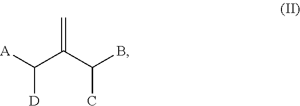

wherein:
A and B are combinations of: COOH, COOR, COCl, CONR′R″, COSR, (CO)O(CO)R′, COH, CNR′R″, CN, CX (X=Cl, Br, I), CHO, H, CH₃, NCO, CSR;
C=Halogen, OH, H, R, NR′R″, O (oxirane), SR, OR;
D=Halogen, OH, H, R, NR′R″, O (oxirane), SR, OR; and
R, R′, R″ are any combination of =H, alkyl, aryl, carbocycle.

In another such aspect, the product may be represented by Formula III:

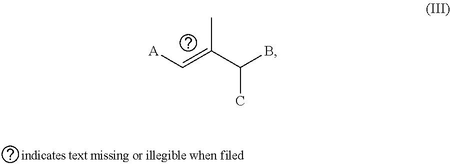

wherein:
A and B are combinations of: COOH, COOR, COCl, CONR′R″, COSR, (CO)O(CO)R′, COH, CNR′R″, CN, CX (X=Cl, Br, I), CHO, H, CH₃, NCO, CSR;
C=Halogen, OH, H, R, NR′R″, O (oxirane), SR, OR; and
R, R′, R″ are any combination of =H, alkyl, aryl, carbocycle.

In another such aspect, the product may be represented by Formula IV:

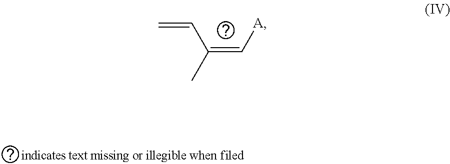

wherein:
A is one of: COOH, COOR, COCl, CONR′R″, COSR, (CO)O(CO)R′, COH, CNR′R″, CN, CX (X=Cl, Br, I), CHO, H, CH₃, NCO, CSR; and
R, R′, R″ are any combination of =H, alkyl, aryl, carbocycle.

In one particular such aspect, the target compound is 3MPD:

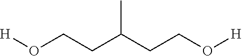

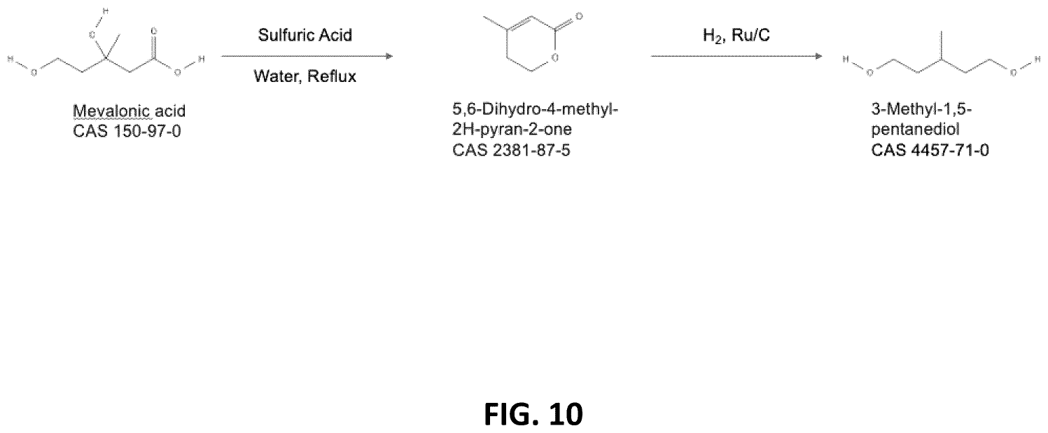

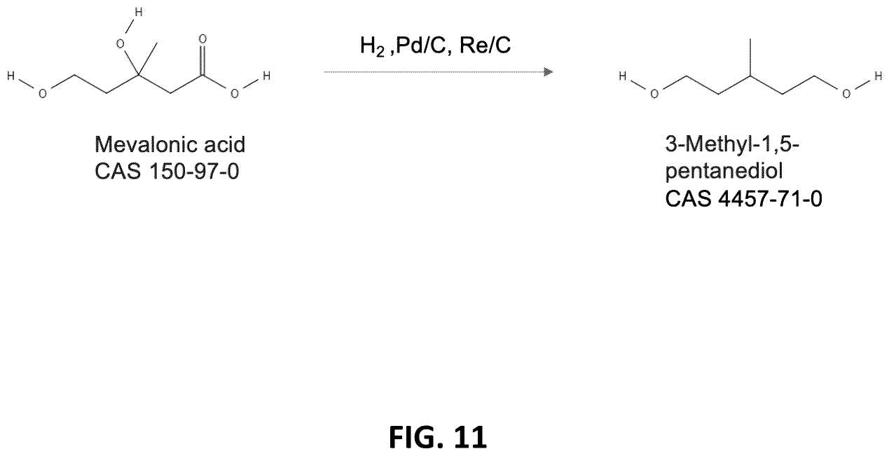

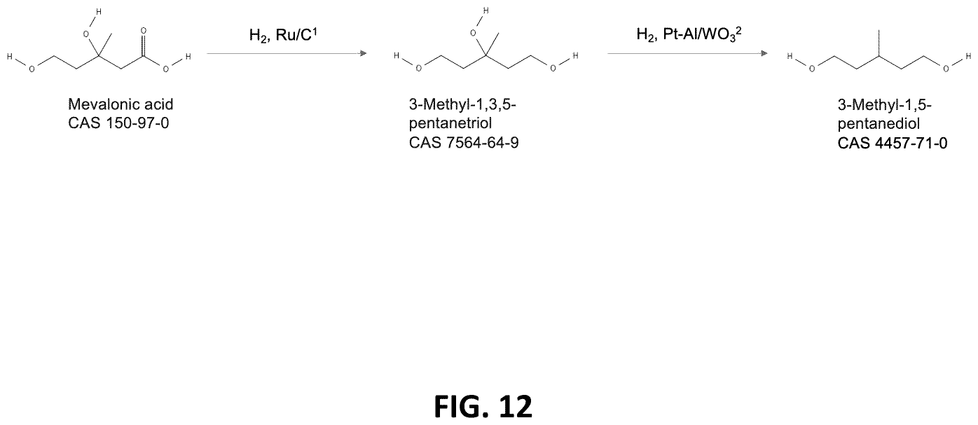

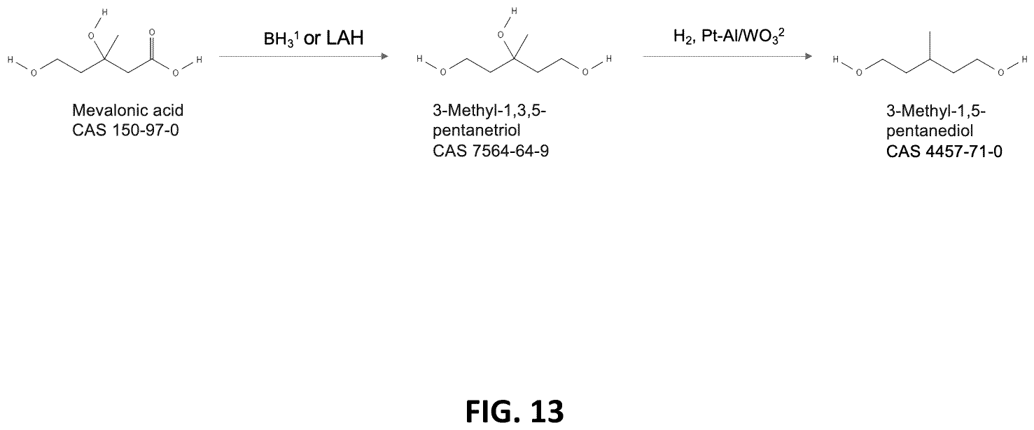

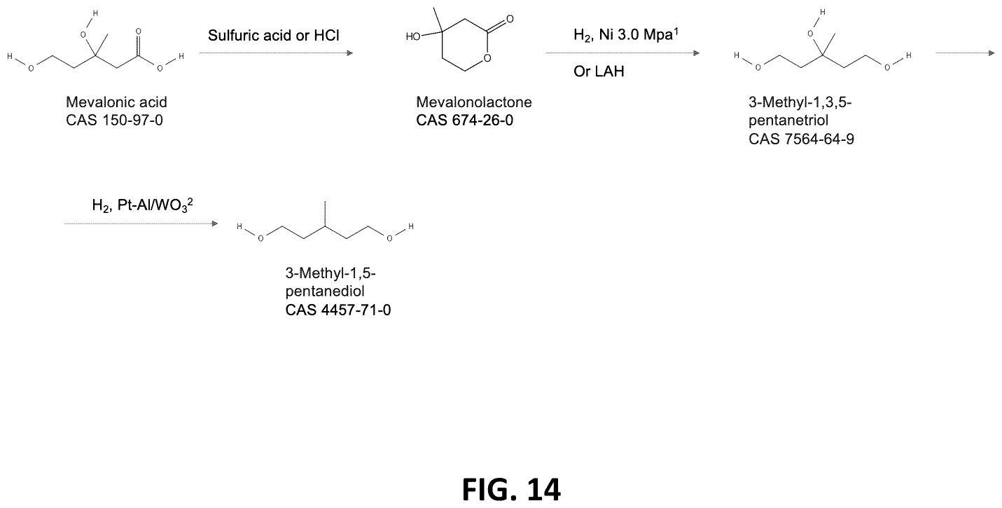

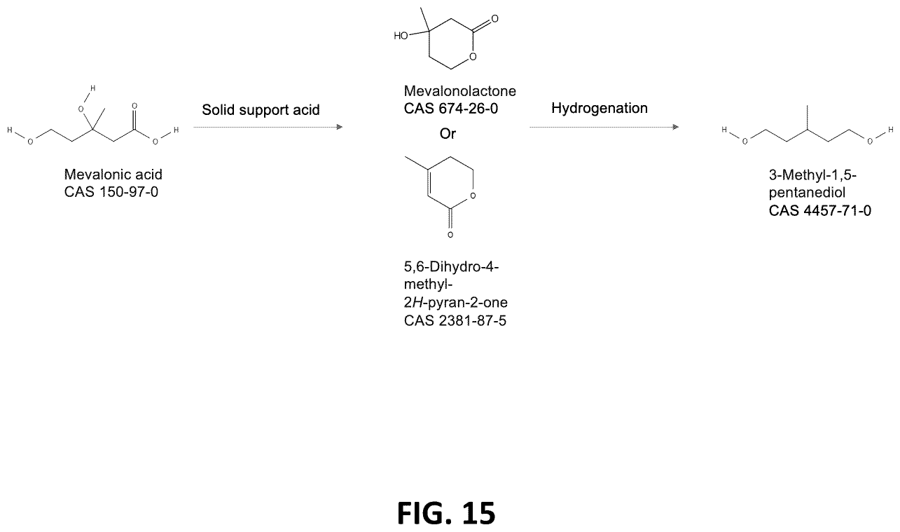

Several chemical conversion routes are readily apparent to transform MVA to 3MPD. By way of example only, in one aspect, shown in FIG. 10, MVA may be dehydrated to form AMVL. AMVL may be catalytically hydrogenated to form 3MPD. In another aspect, shown in FIG. 11, MVA may be directly catalytically hydrogenated and reduced to form 3MPD. In other aspects, shown in FIG. 12 and FIG. 13, MVA maybe reduced to form 3-methyl-1,3,5-pentanetriol (CAS 7564-64-9). The triol may be deoxygenated to form 3MPD. In another aspect, shown in FIG. 14, MVA may be dehydrated to form MVL. The lactone ring maybe opened, and the resulting 3-methyl-1,3,5-pentanetriol may be catalytically hydrogenated to form 3MPD. In yet another aspect, shown in FIG. 15, MVA may be dehydrated either to MVL or AMVL, followed in either case by hydrogenation to form 3MPD. Other synthetic routes from MVA to 3MPD will be readily envisioned by the skilled person.

In another aspect, the target compound is 3-methylenepentanedioic acid (CAS 90423-57-7):

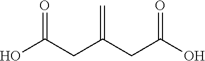

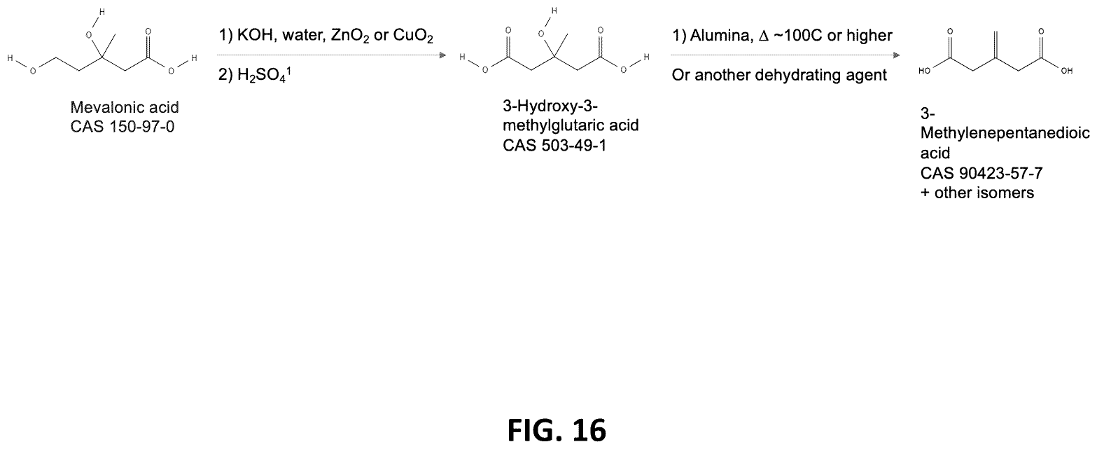

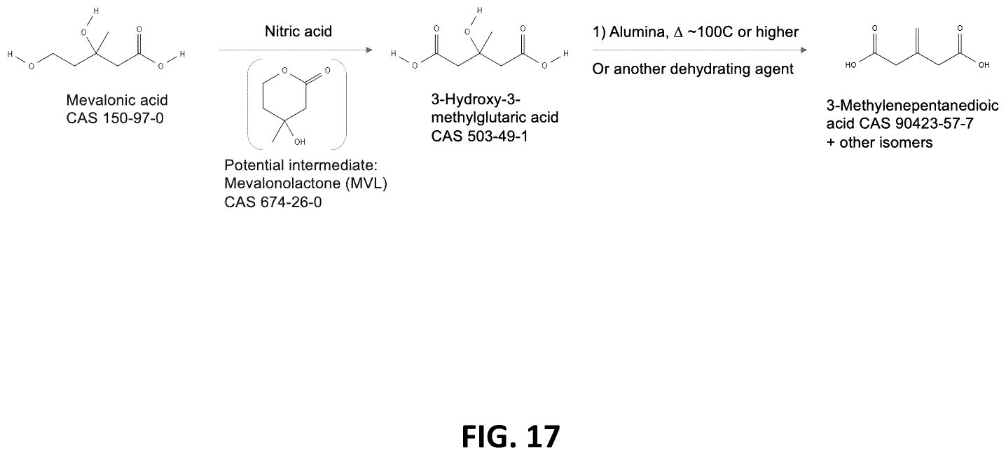

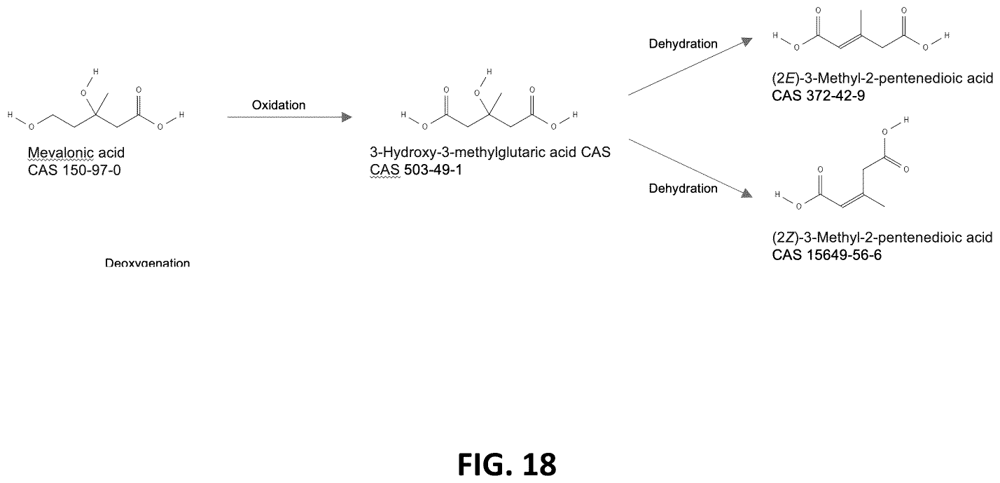

Several chemical conversion routes are readily apparent to transform MVA to 3-methylenepentanedioic acid. By way of example only, in two different aspects, shown in FIG. 16 and FIG. 17, respectively, MVA may be oxidized to form 3-hydroxy-3-methylglutaric acid (CAS 503-49-1). 3-hydroxy-3-methylglutaric acid may be dehydrated to form 3-methylenepentanedioic acid. Alternatively, as shown in FIG. 18, 3-hydroxy-3-methylglutaric acid may be dehydrated to form isomers (2E)-3-methyl-2-pentenedioic acid (CAS 372-42-9) and (2Z)-3-methyl-2-pentenedioic acid (CAS 15649-56-6). Any of the three isomers may be further reduced to form 3-methylglutaric acid (CAS 626-51-7) (not shown).

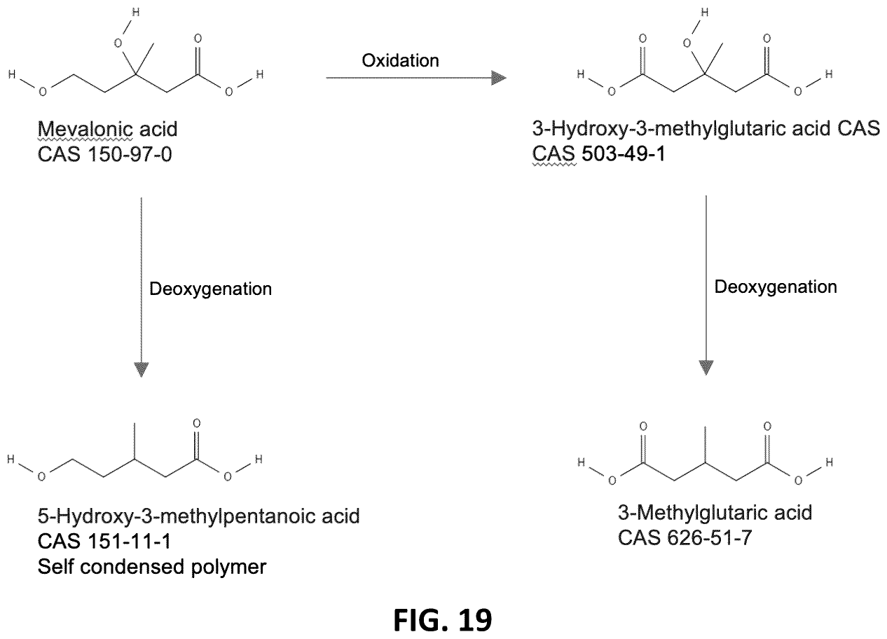

Alternatively still, as shown in FIG. 19, MVA or 3-hydroxy-3-methylglutaric acid may by deoxygenated to form 5-hydroxy-3-methylpentanoic acid (CAS 151-11-1) or 3-methylglutaric acid, respectively.

In another aspect, MVA is first transformed into a lactone and then further transformed into commercially valuable downstream products. In one such aspect, the mevalonate lactone intermediate may be represented by Formula V:

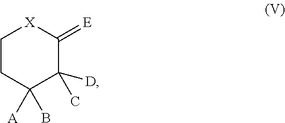

wherein:
X=O or N;
A, B, C, and D are combinations of H, R, OH, OR, O (oxirane), SR, CRS=O, NR′R″, Halogen; and
E=O, S, N=R.

In another such aspect, the mevalonate lactone intermediate may be represented by Formula VI:

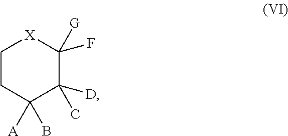

wherein:
X=O or N;
A, B, C, and D are combinations of H, R, OH, OR, O (oxirane), SR, CRS=O, NR′R″, Halogen; and
F and G are combinations of: H, R, CR, OH, OR, O (oxirane), CRS=O, NR′R″, Halogen.

In another such aspect, the mevalonate lactone intermediate may be represented by Formula VII:

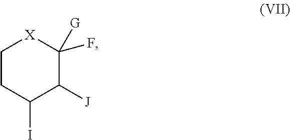

wherein:
X=O or N;
F and G are combinations of: H, R, CR, OH, OR, O (oxirane), CRS=O, NR′R″, Halogen; and
I and J are combinations of: H, R, OR, Halogen.

In another such aspect, the mevalonate lactone intermediate may be represented by Formula VIII:

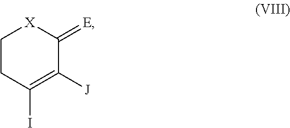

wherein:
X=O or N;
E=O, S, N=R; and
I and J are combinations of: H, R, OR, Halogen.

In another such aspect, the mevalonate lactone intermediate may be represented by Formula IX:

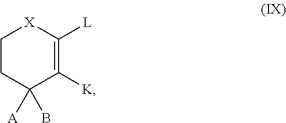

wherein:
X=O or N;
A and B are combinations of H, R, OH, OR, O (oxirane), SR, CRS=O, NR′R″, Halogen;
K=H, R, OR, Halogen; and
L=H, R, CR, OH, OR, CRS=O, NR′R″, Halogen.

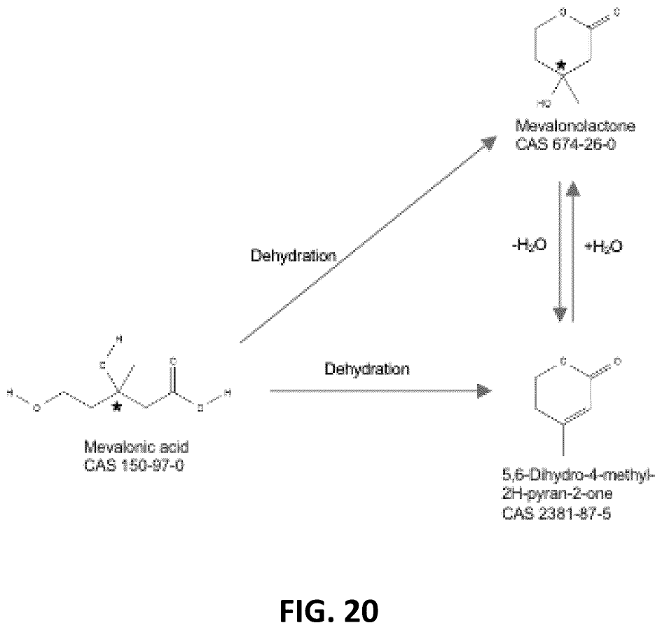

In one particular such aspect, the target compound is MVL. Several chemical conversion routes are readily apparent to transform MVA to MVL. By way of example only, as shown in FIG. 20, MVA may be transformed to MVL via dehydration, directly or through hydrolysis of AMVL.

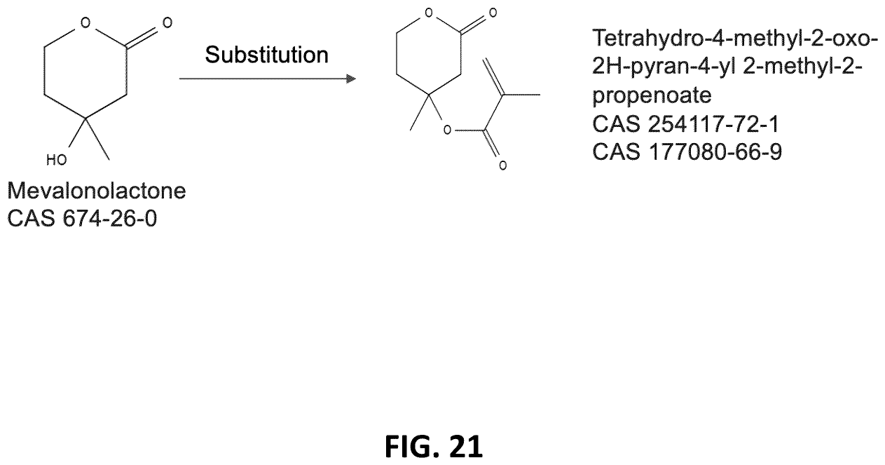

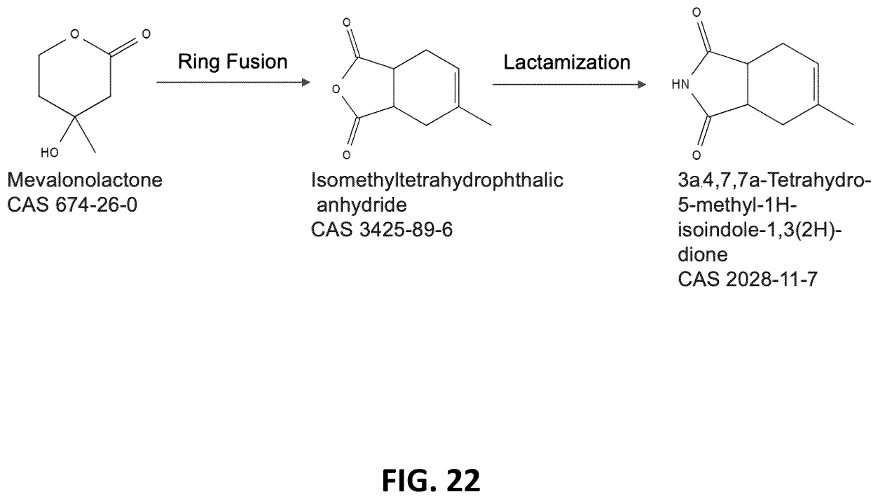

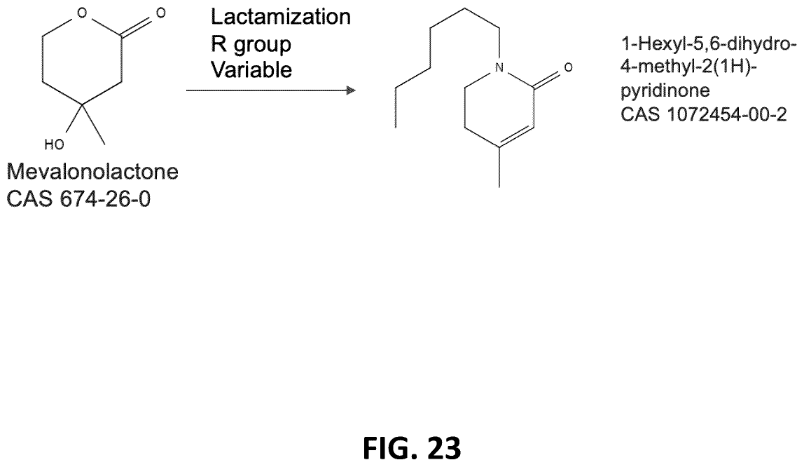

MVL, in turn, may be further transformed, e.g., by substitution (FIG. 21), ring fusion and, optionally, lactamization (FIG. 22), or direct lactamization (FIG. 23).

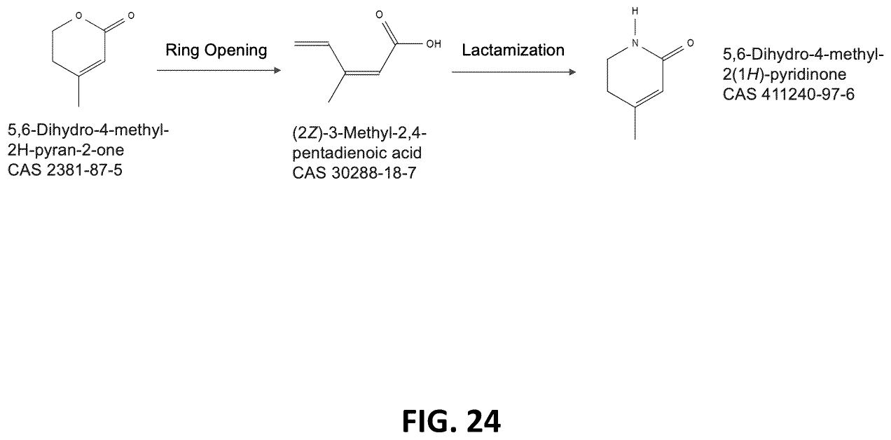

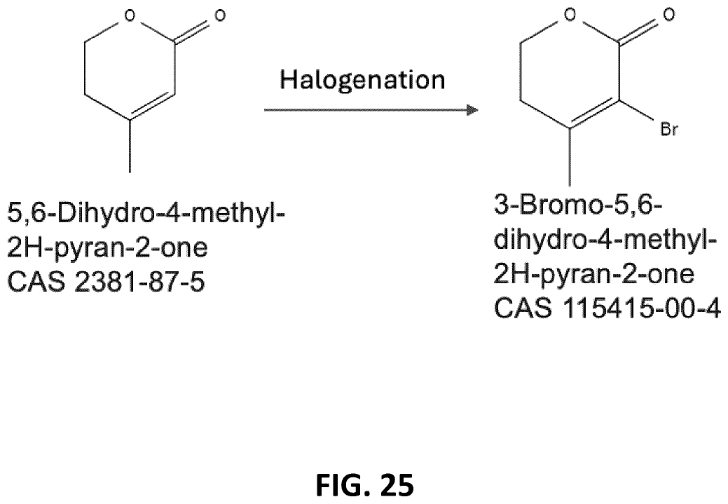

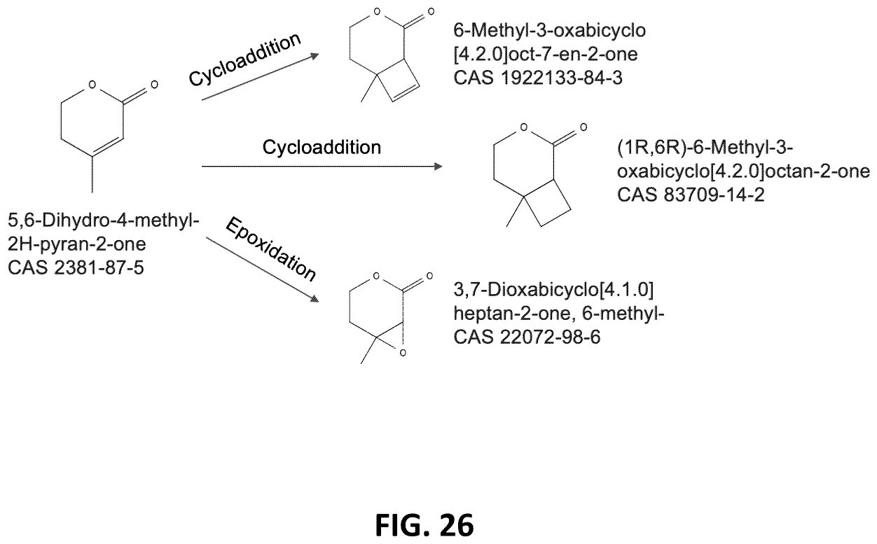

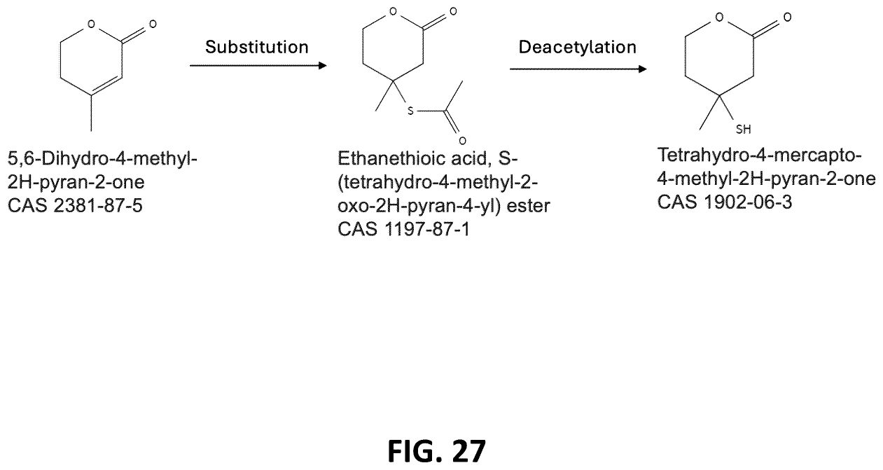

In another such aspect, the target compound is AMVL, the synthesis of which from MVA is shown in, e.g., FIG. 20. AMVL, in turn, may be further transformed into myriad commercial useful compounds. For example, as shown in FIG. 24, AMVL may be subjected to ring opening conditions to form (2Z)-3-methyl-2,4-pentadienoic acid (CAS 30288-18-7) and, optionally, lactamized to form 5,6-dihydro-4-methyl-2(1H)-pyridinone (CAS 411240-97-6). AMVL may be halogenated, as shown in FIG. 25. AMVL may be transformed into a fused ring structure by, e.g., cycloaddition or epoxidation, as shown in FIG. 26. AMVL may also be deacetylated, e.g., as shown in FIG. 27, by substitution followed by deacetylation.

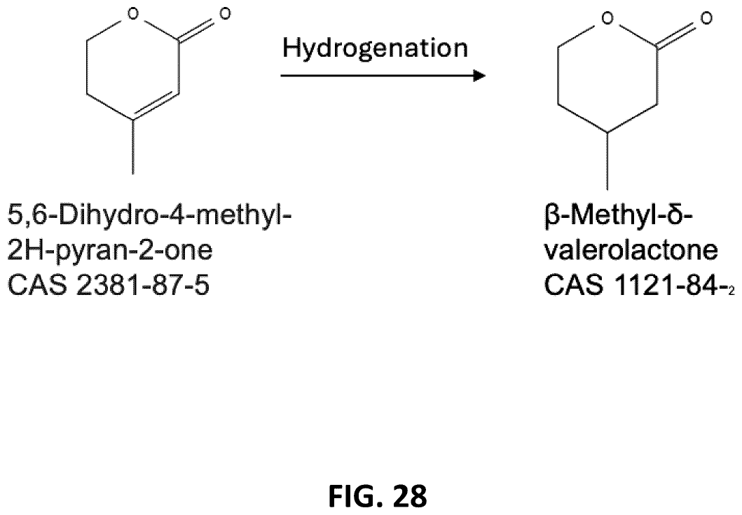

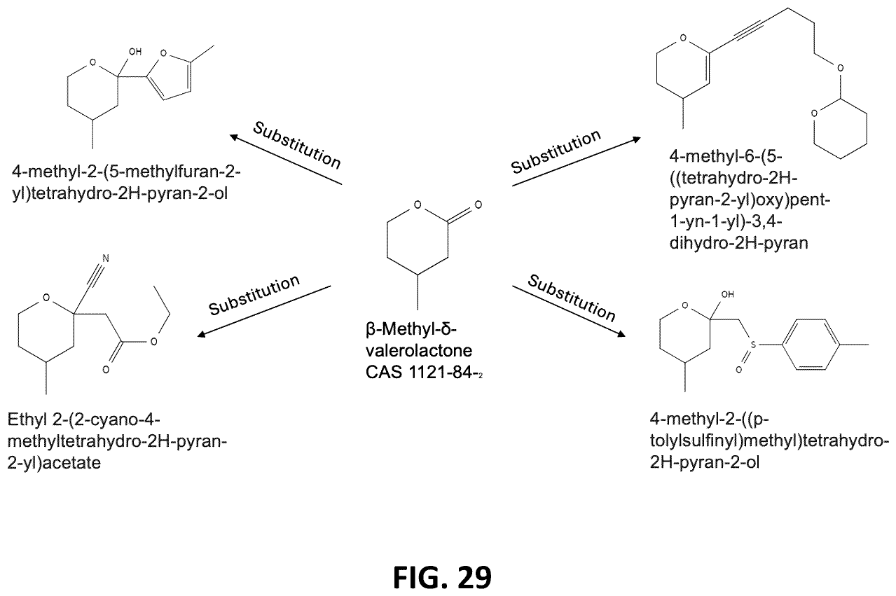

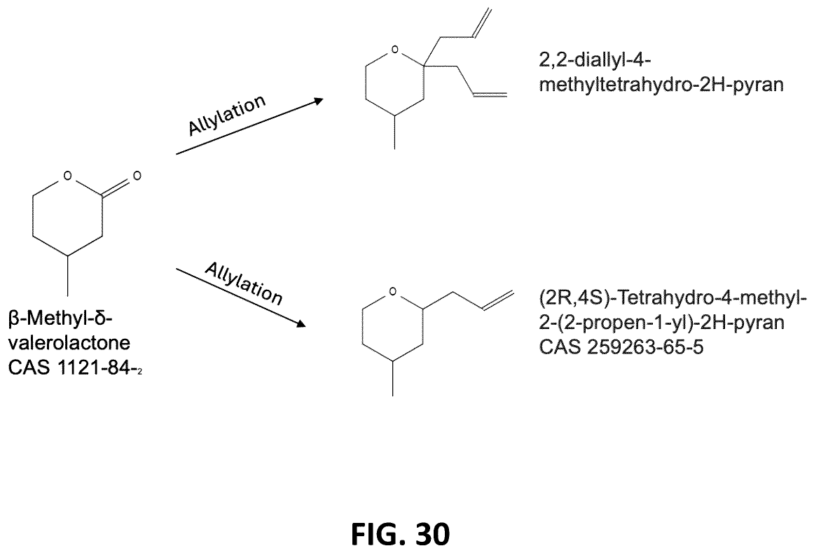

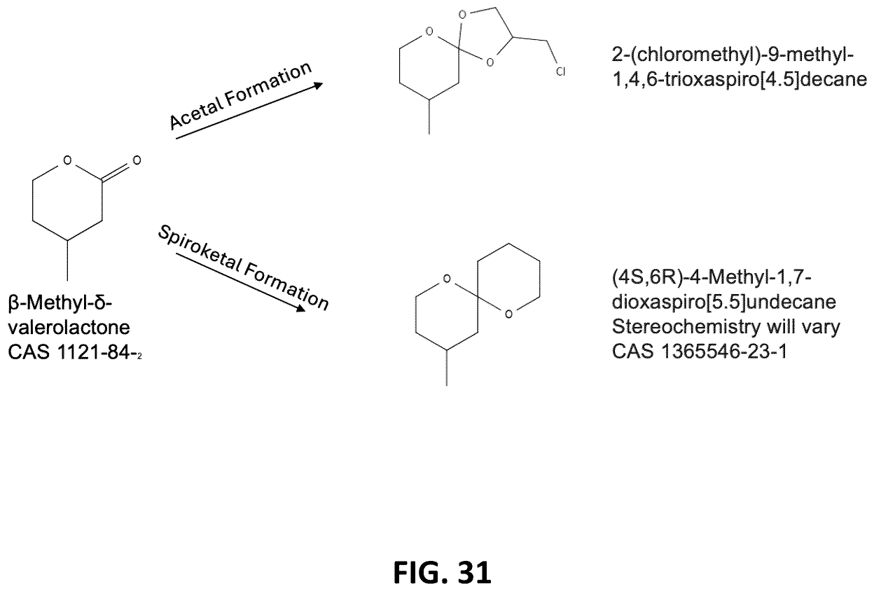

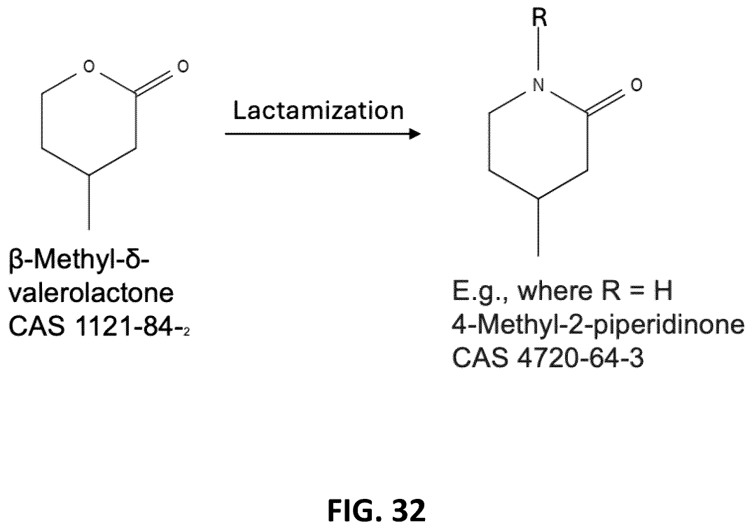

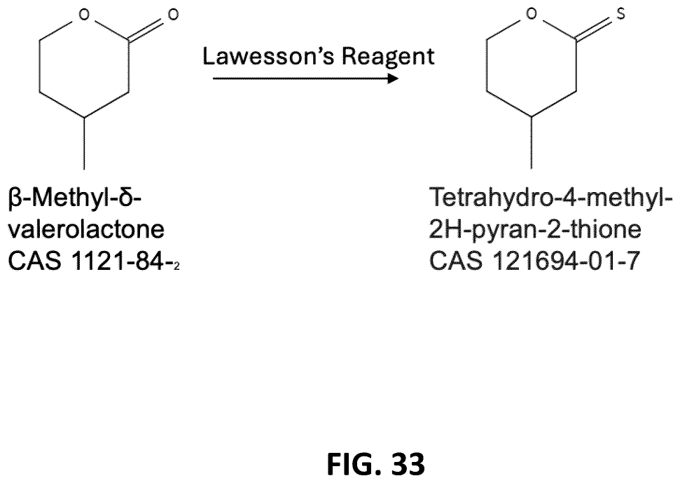

In another particular such aspect, the target compound is β-methyl-δ-valerolactone. By way of example, β-methyl-δ-valerolactone may be formed by hydrogenation of AMVL, as shown in FIG. 28. β-methyl-δ-valerolactone may, in turn, be transformed into several known and novel compounds. For example, as shown in FIG. 29, β-methyl-δ-valerolactone may be subjected to substitution reactions to form, e.g., 4-methyl-2-(5-methylfuran-2-yl)tetrahydro-2H-pyran-2-ol, 4-methyl-6-(5-((tetrahydro-2H-pyran-2-yl)oxy)pent-1-yn-1-yl)-3,4-dihydro-2H-pyran, 4-methyl-2-((p-tolylsulfinyl)methyl)tetrahydro-2H-pyran-2-ol, and ethyl 2-(2-cyano-4-methyltetrahydro-2H-pyran-2-yl)acetate. β-methyl-δ-valerolactone may be allylated, as shown in FIG. 30, to form 2,2-diallyl-4-methyltetrahydro-2H-pyran or (2R,4S)-tetrahydro-4-methyl-2-(2-propen-1-yl)-2H-pyran (CAS 259263-65-5). β-methyl-δ-valerolactone may also undergo acetal or spiroketal reaction conditions to yield spiro compounds, as shown in FIG. 31. β-methyl-δ-valerolactone may also undergo lactamization (as shown in FIG. 32) or may be subjected to Lawesson's Reagent to form the corresponding thione (as shown in FIG. 33). The skilled artisan will readily recognize additional transformations.

### EXAMPLES

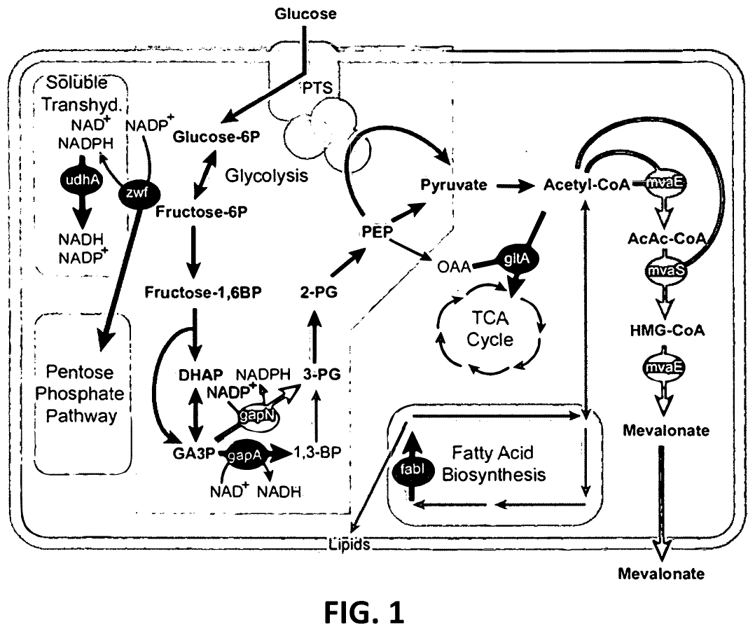

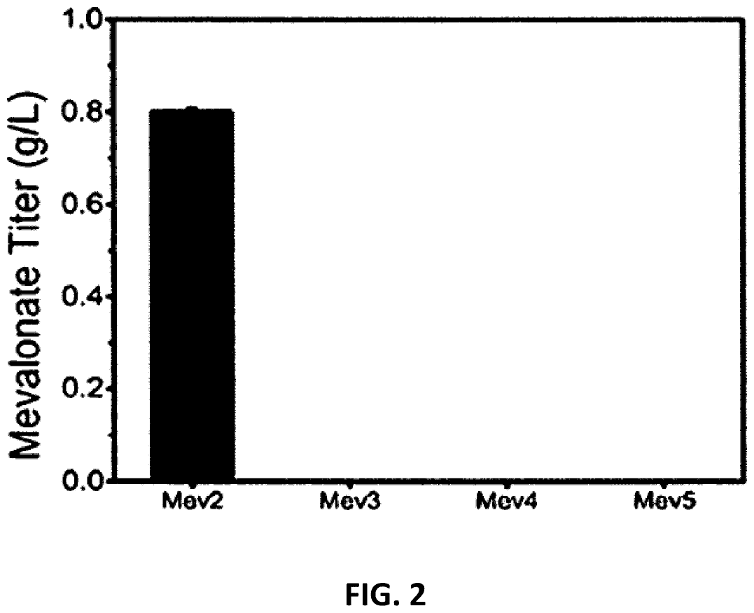

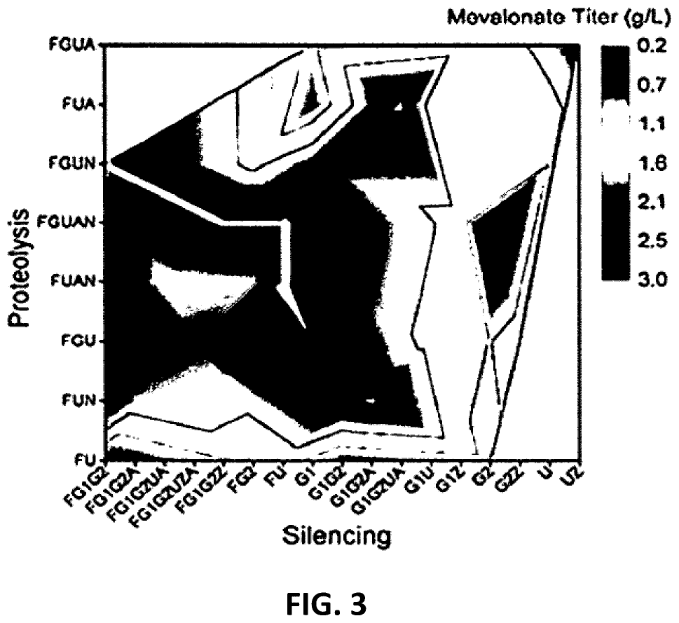

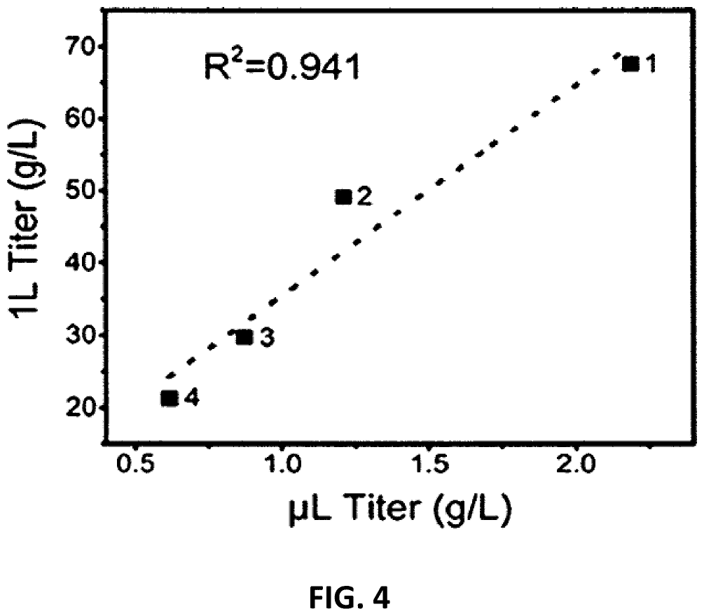

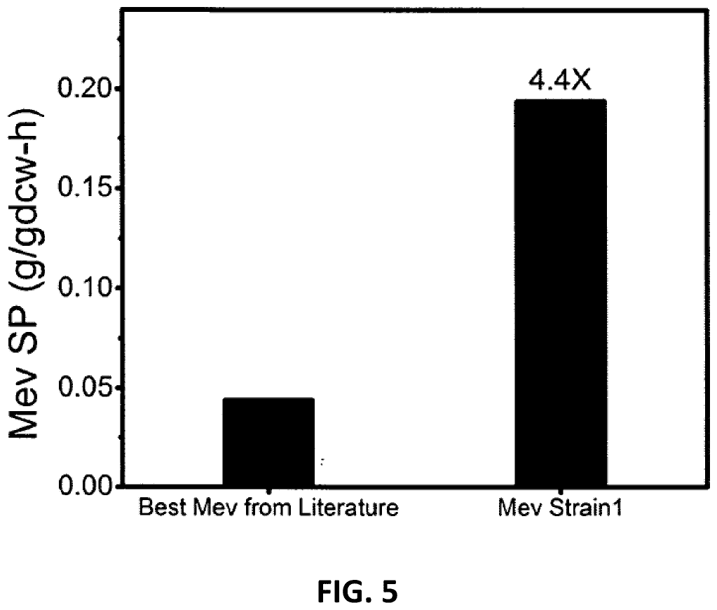

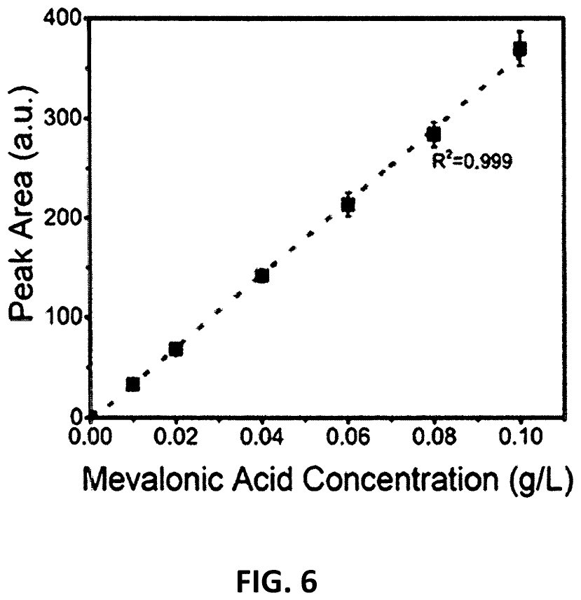

Dynamic production pathways were constructed for mevalonic acid biosynthesis (FIG. 1). In International Application No. PCT/US2018/019040, to which the instant disclosure claims priority, a set of two-gene production pathway plasmids encoding three enzymatic functions was constructed for mevalonate production, consisting of the E. faecalis, mvaE, and mvaS genes encoding a bifunctional acetyl-CoA acetyltransferase, NADPH dependent HMG-CoA reductase, and HMG-CoA synthase, respectively. A mutant mvaS gene, mvaS(A110G), with higher activity was used. Production plasmids were initially evaluated for mevalonate production in the control strain (FIG. 2). The best producing plasmid was introduced into a variety of engineered “Valve” strains and evaluated in micro-fermentations (FIG. 3). A subset of statistically differentiated strains were evaluated in 1 L fermentations to assess scalability (FIG. 4), which was predictive. In some cases, a performing strain produced meaningful titers and yields, e.g., 97 g/L in 78 h of production with a yield of 0.46 g/g (84% of theoretical yield) (FIG. 5). Specific productivity for this mevalonate strain is over 4-fold higher than the best previously reported results (FIG. 6). An improved strain and biosynthesis are disclosed below.

### Example 1—Strain Construction

### Reagents and Media

Unless otherwise stated, all materials and reagents used were of the highest grade possible and purchased from Sigma (St. Louis, Mo.), unless otherwise stated. Luria Broth was used for routine strain and plasmid propagation and construction. Working antibiotic concentrations were as follows: ampicillin (100 μg/mL), kanamycin (35 μg/mL), chloramphenicol (35 μg/mL), spectinomycin (100 μg/mL), zeocin (50 μg/mL), gentamicin (10 μg/mL), blasticidin (100 μg/mL), puromycin (150 μg/mL), tetracycline (5 μg/mL). Luria Broth with low salt (Lennox formulation) was used to select for zeocin, blasticidin, and puromycin resistant clones. In addition, for puromycin selection, phosphate buffer (pH=8.0) was added to LB Lennox to a final concentration of 50 mM.

The oligonucleotides and synthetic linear DNA fragments (Gblocks™) used for strain construction and confirmation were obtained from Integrated DNA Technologies (IDT, Coralville, Iowa). Strain DLF_0047 (DLF_0025, fabl-DAS+4-gentR, zwf-DAS+4-bsdR) was obtained as described in PCT/US2018/019040, see para. [00162] and Table 5. Chromosomal modifications were made using standard recombineering methodologies. The modifications were made by either directly integrating an antibiotic cassette along with the desired modification, as in the case of C-terminal DAS+4 tags carrying antibiotic resistance cassettes, or through scarless tet-sacB selection and counterselection. The recombineering plasmid pSIM5 and the tet-sacB selection/counterselection marker cassette used in constructing the strains were kind gifts from Donald Court (NCI, https://redrecombineering.ncifcrf.gov/court-lab.html). Briefly, the tet-sacB selection/counterselection cassette was amplified using the appropriate oligos supplying \tilde{ }50 bp flanking homology sequences using Econotaq (Lucigen Middleton, Wis.) according to the manufacturer's instructions, with an initial 10-min denaturation at 94° C., followed by 35 cycles at 94° C. for 15 s, 52° C. for 15 s, and 72° C. for 5 min. Cassettes used for “curing” of the tet-sacB cassette or direct integration (when an antibiotic marker is present) were obtained as gBlocks from IDT. Chromosomal modifications were confirmed by PCR amplification and sequencing (Eton Biosciences) using paired oligonucleotides, either flanking the entire region, or in the case of DAS+4 tag insertions, an oligo 5′ of the insertion and one internal to the resistance cassette.

E. coli Plasmid Construction

Gene silencing guide arrays were expressed from pCASCADE plasmid as described in PCT/US2018/019040, see para. [00163]. The single gRNA (pCASCADE-gltA1-gltA2-gapAP1) used in this study was previously constructed in PCT/US2018/019040, see para. [00163]. The sequence of the is gRNA is

(SEQ ID NO. 1)
TCGAGTTCCCCGCGCCAGCGGGGATAAACCGAAAAGCATATAATGCGT
AAAAGTTATGAAGTTCGAGTTCCCCGCGCCAGCGGGGATAAACCGTAT
TGACCAATTCATTCGGGACAGTTATTAGTTCGAGTTCCCCGCGCCAGC
GGGGATAAACCGGTTTTTGTAATTTTACAGGCAACCTTTTATTCGAGT
TCCCCGCGCCAGCGGGGATAAACCG

and was cloned using the primers gapAP1-FOR (SEQ ID NO. 2), pCASCADE-REV (SEQ ID NO. 3), pCASCADE-FOR (SEQ ID NO. 4), and gltA2-REV (SEQ ID NO. 5). The templates were pCASCADE-gapAP1 and pCASCADE-G1G2, see PCT/US2018/019040, p. 69, Table 9. The CASCADE guide array was designed to target CASCADE PAM sites near the −35 or −10 box of the promoter of interest. 30 bp at the 3′ end of PAM site were selected as the guide sequence and cloned into pCASCADE plasmid using QS site-directed mutagenesis (NEB, MA) following the manufacturer's protocol, except that 5% v/v DMSO was added to the Q5 PCR reaction. The PCR was performed with an initial denaturation step at 98° C. for 30 s followed by cycling at 98° C. for 10 s, 72° C. for 30 s, and 72° C. for 1.5 min (the extension rate was 30 second/kb) for 25 cycles, then a final extension for 2 min at 72° C. 2 μL of PCR mixture was used for 10 μL KLD reaction at room temperature for 1 h, after which, 1 μL of the KLD reaction mixture was used in transforming electrocompetent cells.

The method used in constructing the plasmid encoding the mevalonate biosynthetic pathway used in this study was previously described in PCT/US2018/019040, see para. [00163]. Briefly, mevalonate production pathway enzymes were expressed from a high copy number plasmid under the control of low phosphate inducible promoters. The mevalonate pathway gene sequences were codon optimized using the Codon Optimization Tool from the IDT website, and phosphorylated G-blocks were designed and purchased from IDT for each pathway. The plasmid was assembled using NEBuilder® HiFi DNA Assembly Master Mix following the manufacturer's protocol (NEB, MA). pSMART-HC-Kan (Lucigen, WI) was used as backbone for the mevalonate pathway genes. The plasmid sequence was confirmed by DNA sequencing (Eton Bioscience, NC) and deposited with Addgene.

TABLE 1
Oligonucleotides used in strain construction.

| Oligo | SEQ ID NO. |
|---|---|
| iclR_tetA_f | 6 |
| ilcR_sacB_R | 7 |
| iclR_500up | 8 |
| iclR_500dn | 9 |
| sspB_kan_F | 10 |
| sspB_kan_R | 11 |
| sspB_conf_F | 12 |
| sspB_conf_R | 13 |
| cas3_tetA_F | 14 |
| cas3_sacB_R | 15 |
| cas3_conf_F | 16 |
| cas3_500dn | 17 |
| fabI_conf_F | 18 |
| gapA_conf_F | 19 |
| gltA_conf_F | 20 |
| udhA_conf_F | 21 |
| bsdR_intR | 22 |
| gentR_intR | 23 |
| purR_intR | 24 |
| tetA_intR | 25 |
| zeoR_intR | 26 |

TABLE 2
Synthetic DNA used for strain construction.

| Synthetic DNA | SEQ ID NO. |
|---|---|
| tetA-sacB Cassette | 27 |
| ΔiclR-cure | 28 |
| ΔarcA-cure | 29 |
| Δcas3::ugBp-sspB-pro-casA | 30 |
| fabI-DAS + 4-gentR | 31 |
| gltA-DAS + 4-zeoR | 32 |
| udhA-DAS + 4-bsdR | 33 |
| proCp-galP | 34 |
| proCp-glk | 35 |

### Example 2—E. coli Microfermentations

Luria Broth (LB) was used for routine strain and plasmid construction and propagation. LB was obtained by suspending 20 g of LB Broth (Lennox) powder (sigma-Aldrich) in 1 L of distilled water and autoclaved according to the manufacturer's instructions. Antibiotics were supplemented where necessary at the following working concentrations: kanamycin (35 μg/ml) and chloramphenicol (35 μg/ml).

Plasmids were transformed into host strains by electroporation using an Eppendorf Eporator apparatus according to the manufacturer's protocol. After transformation, the cells were recovered for at least one hour in LB outgrowth media and plated on LB agar plates supplemented with the appropriate antibiotics. After 16 h of overnight incubation at 37° C., single colonies were isolated and used for microfermentation experiments.

To start the microfermentation, single colonies from the transformation plate, along with appropriate controls, were inoculated into 150 μl of SM10++ medium supplemented with appropriate antibiotics in a flat bottom 96-well plate (Genesee Scientific, 25-104). The plate was covered with a sandwich cover (Model # CR1596 obtained from EnzyScreen, Haarlam, The Netherlands) to minimize evaporation during the experiment. The 96 well plates were cultured at 37° C., 300 rpm, for 16 h in a shaker of orbit 50 mm.

After 16 h of growth, the cells were pelleted by centrifugation at 4000 rpm for 10 min, and the supernatant was discarded. Cells were washed by resuspension in fresh FGM10.2 no phosphate medium and pelleted. Pellets were resuspended in 50 μl FGM10.2 no phosphate medium supplemented with appropriate antibiotics. 5 μl of the resuspended culture was added to 195 μl of water for OD600 measurement using a standard 96 well plate. The production OD was normalized to OD600=1 in a sterile standard 96 well plate for a total volume of 150 μl, using FGM10.2 no phosphate medium supplemented with appropriate antibiotics. The 96 well plates were covered with sandwich cover (Model # CR1596 obtained from EnzyScreen, Haarlam, The Netherlands) and incubated at 37° C., 300 rpm for 24 h. After 24 h of production, OD600 was recorded, and all samples from each well were pelleted by centrifugation. The supernatants were collected for subsequent analytical measurements. Triplicate microfermentations were performed for each strain tested.

### Fermentation Primary Seed Preparation

A single colony from the transformation plate was inoculated into 5 ml SM10++ culture supplemented with appropriate antibiotics and cultured at 37° C., 300 rpm for 16 h. 500 μl of the SM10++ overnight culture was inoculated into 50 ml SM10 media with appropriate antibiotics in a baffled shake flask. The culture was incubated at 37° C., 300 rpm for 24 h. The culture was harvested by centrifugation at 4000 rpm for 15 min, the supernatant was discarded, and cell culture was normalized to OD600=10 using fresh SM10 media. 25% volume of sterile 50% glycerol was added and briefly vortexed to mix. The primary seeds were saved in 10 ml aliquots in cryogenic vials and stored in −80° C. freezer. 10 ml of primary seed was used for each 6 L fermentation run.

### Media Composition

TABLE 3
SM10++ media composition.

| Ingredient | Final Concentration | |
|---|---|---|
| 10X FGM10 Salt, | 1X | |
| pH 7.5 | (9 g Ammonium Sulfate, | |
| | 0.25 g Citrate) | |
| Phosphate Buffer, | 5.00 | mM |
| pH 6.0 | | |
| Trace Metals | 2X | |
| Fe (II) Sulfate | 0.16 | mM |
| MgSO₄ | 2.50 | mM |
| CaSO₄ | 0.06 | mM |
| Glucose | 45.0 | g/L |
| MOPS, pH 7.4 | 200 | mM |
| Thiamine-HCl | 0.01 | g/L |
| Yeast Extract | 2.5 | g/L |
| Casamino Acids | 2.5 | g/L |

TABLE 4
SM10 media composition.

| Ingredient | Final Concentration | |
|---|---|---|
| 10X FGM10 Salt, | 1X | |
| pH 7.5 | (9 g Ammonium Sulfate, | |
| | 0.25 g Citrate) | |
| Phosphate Buffer, | 5.00 | mM |
| pH 6.0 | | |
| Trace Metals | 2X | |
| Fe (II) Sulfate | 0.16 | mM |
| MgSO₄ | 2.50 | mM |
| CaSO₄ | 0.0625 | mM |
| Glucose | 45.0 | g/L |
| MOPS, pH 7.4 | 200 | mM |
| Thiamine-HCl | 0.01 | g/L |
| Yeast Extract | 1.0 | g/L |

TABLE 5
FGM10.2 media composition.

| Ingredient | Final Concentration | |
|---|---|---|
| 10X FGM10 Salt, | 1X | |
| pH 7.5 | (9 g Ammonium Sulfate, | |
| | 0.25 g Citrate) | |
| Trace Metals | 2X | |
| Fe (II) Sulfate | 0.16 | mM |
| MgSO₄ | 10 | mM |
| CaSO₄ | 0.06 | mM |
| Glucose | 25 | g/L |
| MOPS | 200 | mM |
| Thiamine-HCl | 0.01 | g/L |

TABLE 6
Strains used in mevalonate production.

| Host | | Pathway | Silencing guide |
|---|---|---|---|
| strain | Genotype | plasmid | plasmid |
| DLF_0047 | F-, λ-, Δ(araD-araB)567, | PID_233: | PID_301: |
| | lacZ4787(del)(::rrnB-3), rph-1, Δ(rhaD- | pSMART- | pCASCADE- |
| | rhaB)568, hsdR514, ΔackA-pta, ΔpoxB, | yibDp1-mvaE- | gltA1-gltA2- |
| | ΔpflB, ΔldhA, ΔadhE, ΔsspB, ΔiclR, ΔarcA, | phoBp2-mvaS- | gapAp1 |
| | Δcas3::tm-ugpb-sspB-pro [casA*] fabI- | A110G | |
| | das + 4-gentR, gltA-das + 4-zeoR, udhA- | | |
| | das + 4-bsdR | | |
| DLF_0289 | F-, λ-, Δ(araD-araB)567, | PID_233: | None |
| | lacZ4787(del)(::rrnB-3), rph-1, Δ(rhaD- | pSMART- | |
| | rhaB)568, hsdR514, ΔackA-pta, ΔpoxB, | yibDp1-mvaE- | |
| | ΔpflB, ΔldhA, ΔadhE, ΔsspB, ΔiclR, ΔarcA, | phoBp2-mvaS- | |
| | Δcas3::tm-ugpb-sspB-pro [casA*] | A110G | |
| | ΔptsG::proCp-glk, proCp-galP gltA- | | |
| | das + 4::zeoR, udhA-das + 4::bsdR | | |

### Example 3—Production of Mevalonate with 2-Stage Fermentation

### Stock Solutions

TABLE 7
Fraction A1: 10X Salts Stock.

| | | | Total needed |
|---|---|---|---|
| Raw | | Concentration | (g or mL) |
| material | Formula | (g/L) | for 1 L |
| Ammonium Sulfate | (NH₄)₂SO₄ | 90 | 90 |
| Citric Acid | C₆H₈O₇ | 2.5 | 2.5 |
| Anhydrous | | | |
| Magnesium Sulfate | MgSO₄*7H₂O | 24.65 | 24.65 |
| Heptahydrate | | | |
| Calcium Sulfate | CaSO₄*2H₂O | 0.11 | 0.11 |
| Dihydrate | | | |

The components were dissolved in 800 ml demineralized water. The volume was adjusted to 1 L with demineralized water and filter sterilized (filters with 0.2 μm pore size).

TABLE 8
Fraction A2: 5000X TE Stock.

| | | | Total needed |
|---|---|---|---|
| Raw | | Concentration | (g or mL) |
| material | Formula | (g/L) | for 1 L |
| Sulfuric Acid 96.5% | H₂SO₄ | 50 | 50 ml |
| Cobalt sulfate | CoSO₄7•H₂O | 6 | 6 |
| Heptahydrate | | | |
| Copper Sulfate | CuSO₄5•H₂O | 5 | 5 |
| Pentahydrate | | | |
| Zinc Sulfate | ZnSO₄•H₂O | 6 | 6 |
| Monohydrate | | | |
| Sodium Molybdate | Na₂MoO₄2•H₂O | 2 | 2 |
| Dihydrate | | | |
| Boric Acid | H₃BO₃ | 1 | 1 |
| Manganese Sulfate | MnSO₄2•H₂O | 3 | 3 |
| Dihydrate | | | |

The components were dissolved in 800 ml demineralized water. The volume was adjusted to 1 L with demineralized water and filter sterilized (filters with 0.2 μm pore size).

TABLE 9
Fraction A3: 100 mM Iron Sulfate stock solution.

| Raw | | Concentration | Total needed |
|---|---|---|---|
| material | Formula | (g/L) | (g) for 1 L |
| Sulfuric Acid 96.5% | H₂SO₄ | 25 | 25 mL |
| Ferrous Sulfate | Fe(II)SO₄7•H₂O | 27.8 | 27.8 |
| Heptahydrate | | | |

The components were dissolved in 800 ml demineralized water. The volume was adjusted to 1 L with demineralized water and filter sterilized (filters with 0.2 μm pore size).

TABLE 10
Fraction B1: Glucose Feed.

| Raw | | Concentration | Total needed |
|---|---|---|---|
| material | Formula | (g/L) | (g) for 1 L |
| Glucose | C₆H₁₂O₆ | 500 | 500 |

400 ml of the component is dissolved in hot (60° C.) demineralized water. The volume was adjusted to 1 L with demineralized water. The 50% glucose solution was sterilized for 20 min at 121° C.

TABLE 11
Fraction B2: 1M Potassium Phosphate Buffer.

| Raw | | Concentration | Total needed |
|---|---|---|---|
| material | Formula | (g/L) | (g) for 1 L |
| Potassium | KH₂PO₄ | 118.13 | 118.13 |
| Phosphate | | | |
| Monobasic | | | |
| Potassium | K₂HPO₄ | 22.99 | 22.99 |
| Phosphate | | | |
| Dibasic | | | |

The components were dissolved in 600 ml demineralized water. The volume was adjusted to 1 L with demineralized water and filter sterilized (filters with 0.2 μm pore size).

TABLE 12
Fraction B3: Thiamine•HCl stock solution.

| Raw | | Concentration | Total needed |
|---|---|---|---|
| material | Formula | (g/L) | (g) for 10 ml |
| Thiamine•HCl | C₁₂H₁₇ClN₄OS•HCl | 50 | 0.5 |

The component was dissolved in 10 ml demineralized water and filter sterilized (filters with 0.2 μm pore size).

TABLE 13
Fraction B4: Kanamycin stock solution.

| Raw | | Concentration | Total needed |
|---|---|---|---|
| material | Formula | (g/L) | (g) for 50 ml |
| Kanamycin | C₁₈H₃₆N₄O₁₀•H₂SO₄ | 35 | 1.75 |
| Sulfate | | | |

The component was dissolved in 50 ml demineralized water and filter sterilized (filters with 0.2 μm pore size).

TABLE 14
Base Titrant: 10M Ammonium Hydroxide.

| Raw | | Concentration | Total needed |
|---|---|---|---|
| material | Formula | (g/L) | (g) for 1 L |
| DI H₂O | N/A | N/A | 310 mL |
| Ammonium | NH₄OH | 14.5M | 690 mL |
| Hydroxide 28% | | | |

The demineralized water fraction was sterile filtered into a sterile bottle. The ammonium hydroxide fraction was added for a final concentration of 10 M.

TABLE 15
Acid Titrant: 0.5M Hydrochloric acid.

| Raw | | Concentration | Total needed |
|---|---|---|---|
| material | Formula | (g/L) | (g) for 1 L |
| DI H₂O | N/A | N/A | 958.33 mL |
| Hydrochloric | HCl | 12M | 41.67 mL |
| Acid 37% | | | |

The demineralized water fraction was sterile filtered into a sterile bottle. The hydrochloric acid fraction was added for a final concentration of 0.5 M.

TABLE 16
Fermentation Production Media.

| | | | | Final | |
|---|---|---|---|---|---|
| Raw | | Amt. | | Concentration | |
| material | Formula | for 1 L | | (mL/L) | Fraction |
| 10X Salts | N/A | 100 | mL | 100 | A1 |
| 5000X TE stock | N/A | 0.4 | mL | 0.4 | A2 |
| Iron Sulfate | N/A | 1.6 | mL | 1.6 | A3 |
| stock | | | | | |
| DI Water | N/A | to 942.6 mL | | N/A | A4 |
| 500 g/L Glucose | N/A | 50 | mL | 25 g/L | B1 |
| Phosphate | NA | 6.2 | mL | 6.2 | B2 |
| buffer stock | | | | | |
| Thiamine | NA | 0.20 | mL | 0.2 | B3 |
| Hydrochloride | | | | | |
| stock | | | | | |
| Kanamycin | NA | 1 | mL | 1 | B4 |
| Sulfate | | | | | |
| stock | | | | | |
| Antifoam | NA | 0.083 | mL | 0.083 | C1 |

The components of fraction A were dissolved in 0.8 L demineralized water. Demineralized water was added to the medium to a total of 942.6 mL. The medium was steam sterilized in the fermenter for 20 min at 121° C. The components of fraction B were batched together in the order given and filter sterilized (0.2 um pore size). After cooling to room temperature, fraction B was aseptically added to the sterile batch medium.

The final glucose concentration is 25 g/L, requiring 50 ml of 500 g/L glucose feed per L of media. The starting pH of the production medium fraction A was ˜3.2 and rose to ˜3.4 after addition of fraction B. pH was walked up to the process setpoint, with completion of a process calibration at ˜pH 6. After this, the pH is set to 6.8

Fraction C (antifoam) was added after the media reached pH 6.8; then a 1-point (100%) DO calibration was performed.

### Process Controls

TABLE 17
Fermentation parameters.

| Parameter | Value |
|---|---|
| Media | Minimal media formulation |
| | targeting low biomass |
| pH | 6.8 ± 0.2 |
| Temperature | 37 ± 0.5° C. |
| Glucose Feed | 500 g/L |
| Base | 10M NH₄OH (19%) |
| Acid | 0.5M HCl |
| Dissolved oxygen | 25 ± 5% |
| set point | |
| Inoculation | 0.3% v/v |
| ratio | |

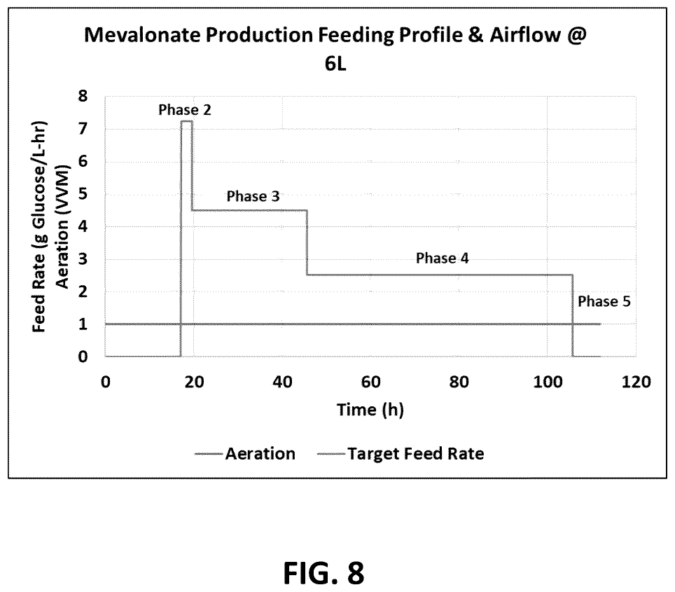

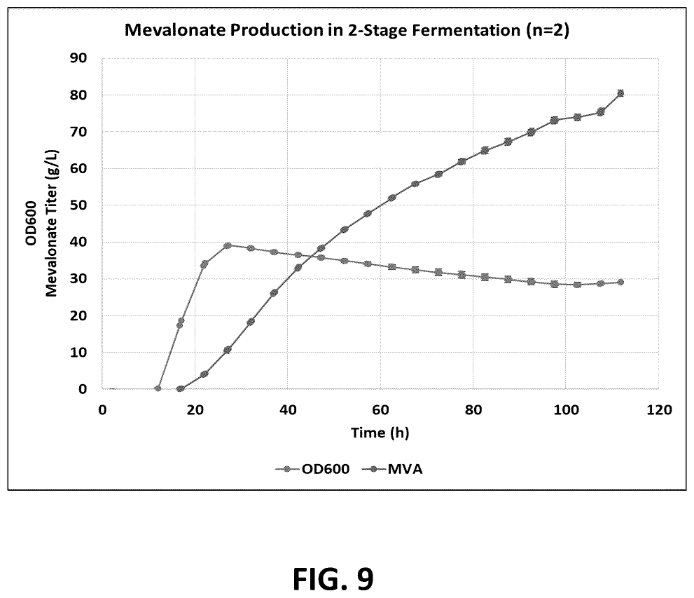

FIG. 8 shows an example mevalonate production feeding/aeration profile. FIG. 9 shows an example mevalonate production fermentation plot.

1. A method for preparing a mevalonate derivative represented by one of the following formulas: 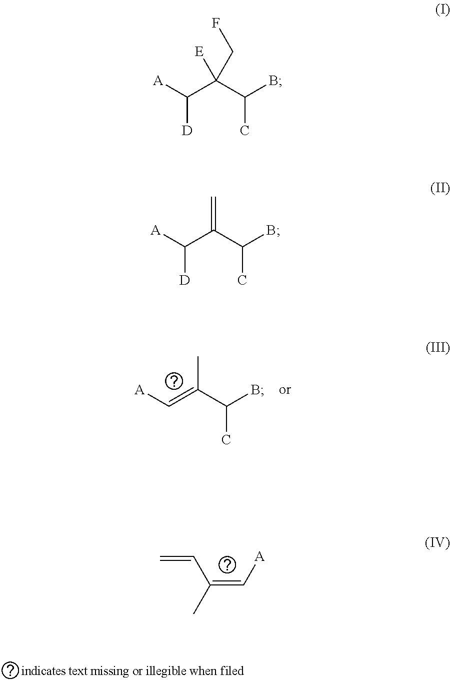
wherein:
A and B are combinations of: COOH, COOR, COCl, CONR′R″, COSR, (CO)O(CO)R′, COH, CNR′R″, CN, CX (X=Cl, Br, I), CHO, H, CH3, NCO, CSR;
C=Halogen, OH, H, R, NR′R″, O (oxirane), SR, OR;
D=Halogen, OH, H, R, NR′R″, O (oxirane), SR, OR;
E=Halogen, OH, H, R, NR′R″, O (oxirane), SR, OR;
F=Halogen, OH, H, R, NR′R″, O (oxirane), SR, OR; and
R, R′, R″ are any combination of =H, alkyl, aryl, carbocycle,
the method comprising:
providing a genetically modified E. coli for producing mevalonate;
growing the genetically modified E. coli in a media in a growth phase, the genetically modified E. coli comprising:
(i) a production pathway comprising at least one production enzyme for biosynthesis of mevalonate; and
(ii) one or more synthetic metabolic valves for reducing or eliminating flux through multiple metabolic pathways within the genetically modified E. coli when the synthetic metabolic valves are induced, the one or more synthetic metabolic valves comprising:
(a) at least one silencing synthetic metabolic valve that silences gene expression of two gene encoding two silenceable enzymes that are selected from fabI, gltA, ldp, zwf, or udhA gene, and
(b) at least one proteolytic synthetic metabolic valve that controls proteolysis of a fabI, gltA, ldp, zwf, or udhA enzyme;
transitioning to a productive stationary phase, the transition comprising:
depletion of a limiting nutrient;
inducing the one or more synthetic metabolic valves; and
activation of the production enzyme for biosynthesis of mevalonate;
producing mevalonate; and
acidifying mevalonate to provide mevalonic acid (MVA).

2. The method of claim 1, wherein the enzyme of the mevalonate production pathway is heterologous to the genetically modified E. coli and the heterologous enzyme is one of: an acetyl-CoA acetyltransferase, NADPH dependent HMG-CoA reductase, HMG-CoA synthase, or a combination thereof.

3. The method of claim 1, wherein the heterologous enzyme of mevalonate production is encoded by: E. faecalis mvaE, E. faecalis mvaS, E. faecalis mvaS(A110G), or a combination thereof.

4. (canceled)

5. The method of claim 1, wherein the enzymes silenced by the silencing metabolic valve are fabI, gltA1, and gltA2 and the enzymes that are subject to enzyme degradation by the proteolytic metabolic valve are fab I, gltA, and udhA.

6. The method of claim 1 wherein at least one silencing synthetic metabolic valve is characterized by CRISPR interference of gene expression of a gene that is a fabI, gltA, ldp, zwf, or udhA gene and expression of a CASCADE plasmid comprising an array of guide RNA genes or wherein at least one proteolytic synthetic metabolic valve is characterized by expression of the proteolytic enzyme operably linked to a or C-terminal DAS4 peptide tag and controlled proteolysis of a fabI, gltA, ldp, zwf, or udhA enzyme by the synthetic metabolic valve is selective for the tag by clpXP protease upon induction of sspB chaperone protein.

7. (canceled)

8. (canceled)

9. The method of claim 1, wherein the genetically modified E. coli is characterized by overexpression of a gene resulting in an increase of a NADPH and/or NAD pool in the genetically modified E. coli during the growth phase.

10. The method of claim 1, wherein transitioning to the productive stationary phase is further modulated by at least one of an artificial chemical inducer including tetracycline, anhydrotetracycline, lactose, isopropyl-beta-D1-thiogalactopyranoside (IPTG), arabinose, raffinose, and tryptophan or depletion of a limiting nutrient from the E. coli culture media.

11. (canceled)

12. The method of claim 1, wherein transitioning to the step of entering a productive stationary phase further comprises inducing expression of the enzyme of the production pathway.

13. (canceled)

14. The method of claim 1, further comprising dehydration of MVA to form 5,6-dihydro-4-methyl-2H-pyran-2-one (AMVL) or mevalonolactone (MVL), or
further comprising dehydrogenation of AMVL or MVL to form 3-methyl-1,5-pentanediol (3MPD), or
further comprising dehydrogenation of MVA to form 3MPD, or
further comprising reduction of MVA to form 3-methyl-1,3,5-pentanetriol, or
further comprising deoxygenation of 3-methyl-1,3,5-pentanetriol to form 3MPD, or
further comprising dehydration of MVA to form MVL, or
further comprising lactone ring opening to form 3-methyl-1,3,5-pentanetriol or
further comprising deoxygenation of 3-methyl-1,3,5-pentanetriol to form 3MPD or
further comprising deoxygenation of MVA to form 5-hydroxy-3-methylpentanoic acid.

15.-21. (canceled)

22. The method of any one of claim 1, further comprising oxidation of MVA to form 3-hydroxy-3-methylglutaric acid.

23. The method of claim 22, further comprising dehydration of 3-hydroxy-3-methylglutaric acid to form 3-methylenepentanedioic acid, (2Z)-3-methyl-2-pentenedioic acid, or (2E)-3-methyl-2-pentenedioic acid or further comprising deoxygenation of 3-hydroxy-3-methylglutaric acid to form 3-methylglutaric acid.

24.-25. (canceled)

26. The method of any one of claim 1, further comprising conversion of MVA to a lactone represented by any one of the following formulas: 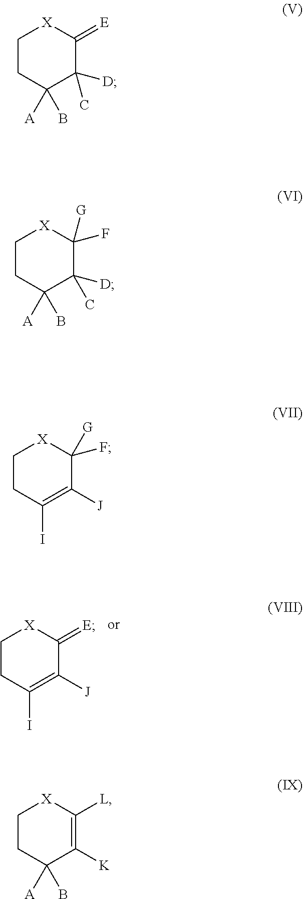
wherein:
X=O or N;
A, B, C, and D are combinations of H, R, OH, OR, O (oxirane), SR, CRS=O, NR′R″, Halogen;
E=O, S, N=R;
F and G are combinations of: H, R, CR, OH, OR, O (oxirane), CRS=O, NR′R″, Halogen;
I and J are combinations of: H, R, OR, Halogen;
K=H, R, OR, Halogen; and
L=H, R, CR, OH, OR, CRS=O, NR′R″, Halogen.

27. The method of claim 26, wherein the lactone is MVL and the method further comprises:
subjecting MVL to a substitution reaction to form tetrahydro-4-methyl-2-oxo-2H-pyran-4-yl 2-methyl-2-propenoate, or
subjecting MVL to a ring fusion reaction to form isomethyltetrahydrophthalic anhydride, or
subjecting MVL to a ring fusion reaction to form isomethyltetrahydrophthalic anhydride and lactamization of isomethyltetrahydrophthalic anhydride to form 3a4,7,7-tetrahydro-5-methyl-1H-isoindole-1,3(2H)-dione, or
lactamization of MVL.

28.-31. (canceled)

32. The method of claim 26, wherein the lactone is AMVL and the method further comprising:
subjecting AMVL to a ring opening reaction to form (2Z)-3-methyl-2,4-pentadienoic acid, or
lactamization of (2Z)-3-methyl-2,4-pentadienoic acid to form 5,6-dihydro-4-methyl-2(1H)-pyridinone, or
halogenation of AMVL, or
cycloaddition to AMVL to form 6-methyl-3-oxabicyclo[4.2.0]oct-7-en-2-one or (1R,6R)-6-methyl-3-oxabicyclo[4.2.0]octan-3-one, or
epoxidation of AMVL to form 3,7-dioxabicyclo[4.1.0]heptan-2-one, 6-methyl- or
subjecting AMVL to a substitution reaction to form ethanethioic acid, S-(tetrahydro-4-methyl-2-oxo-2H-pyran-4-yl) ester,

33.-38. (canceled)

39. The method of claim 32, wherein after subjecting AMVL to a substitution reaction to form ethanethioic acid, S-(tetrahydro-4-methyl-2-oxo-2H-pyran-4-yl) ester, the method further comprises deacetylation of ethanethioic acid, S-(tetrahydro-4-methyl-2-oxo-2H-pyran-4-yl) ester to form tetrahydro-4-mercapto-4-methyl-2H-pyran-2-one.

40. The method of claim 32, further comprising hydrogenation of AMVL to form β-methyl-δ-valerolactone.

41. The method of claim 40, further comprising subjecting β-methyl-δ-valerolactone to a substitution reaction to form one of 4-methyl-2-(5-methylfuran-2-yl)tetrahydro-2H-pyran-2-ol, 4-methyl-6-(5-((tetrahydro-2H-pyran-2-yl)oxy)pent-1-yn-1-yl)-3,4-dihydro-2H-pyran, 4-methyl-2-((p-tolylsulfinyl)methyl)tetrahydro-2H-pyran-2-ol, or ethyl 2-(2-cyano-4-methyltetrahydro-2H-pyran-2-yl) acetate, or
further comprising subjecting β-methyl-δ-valerolactone to an allylation reaction to form one of 2,2-diallyl-4-methyltetrahydro-2H-pyran or (2R,4S)-tetrahydro-4-methyl-2-(2-propen-1-yl)-2H-pyran, or
further comprising subjecting β-methyl-δ-valerolactone to one of an acetal or a spiroketal reaction to form 2-(chloromethyl)-9-methyl-1,4,6-trioxaspiro[4.5]decane or 4-methyl-1,7-dioxaspiro[5.5]undecane, respectively, or
further comprising lactamization of β-methyl-δ-valerolactone, or
further comprising subjecting β-methyl-δ-valerolactone to Lawesson's Reagent to form tetrahydro-4-methyl-2H-pyran-2-thione.

42.-45. (canceled)
